SEQUENCE LISTING (36 sequences)

SEQ ID NO: 1 - DNA, 26 bp, synthetic construct
  misc_feature 1..26: note = NOSterSpeI-F
  source 1..26: mol_type = other DNA
atactagtgaatttccccgatcgttc

SEQ ID NO: 2 - DNA, 28 bp, synthetic construct
  misc_feature 1..28: note = NOSterHindIII-R primer
  source 1..28: mol_type = other DNA
ataagcttagttagctcactcattaggc

SEQ ID NO: 3 - DNA, 31 bp, synthetic construct
  misc_feature 1..31: note = AtESRAscIF3 primer
  source 1..31: mol_type = other DNA
atggcgcgccatggaaaaagccttgagaaac

SEQ ID NO: 4 - DNA, 25 bp, synthetic construct
  misc_feature 1..25: note = AtESRBamHR3 primer
  source 1..25: mol_type = other DNA
atggatcctatccccacgatcttcg

SEQ ID NO: 5 - DNA, 29 bp, synthetic construct
  misc_feature 1..29: note = AtWIND1AscIF3
  source 1..29: mol_type = other DNA
atggcgcgccatggcagctgctatgaatt

SEQ ID NO: 6 - DNA, 27 bp, synthetic construct
  misc_feature 1..27: note = AtWIND1BamHR3 primer
  source 1..27: mol_type = other DNA
atggatcctaagctagaatcgaatccc

SEQ ID NO: 7 - DNA, 23 bp, synthetic construct
  misc_feature 1..23: note = 35S-AatII-F
  source 1..23: mol_type = other DNA
agacgtctctgccgacagtggtc

SEQ ID NO: 8 - DNA, 31 bp, synthetic construct
  misc_feature 1..31: note = NOSterMluI-R primer
  source 1..31: mol_type = other DNA
taacgcgtagttagctcactcattaggcacc

SEQ ID NO: 9 - DNA, 25 bp, synthetic construct
  misc_feature 1..25: note = AmUBO primer
  source 1..25: mol_type = other DNA
cgtgaagaccttgactgggaagacg

SEQ ID NO: 10 - DNA, 26 bp, synthetic construct
  misc_feature 1..26: note = AmUBO primer
  source 1..26: mol_type = other DNA
caaggtgaagagtcgactccttctgg

SEQ ID NO: 11 - DNA, 20 bp, synthetic construct
  misc_feature 1..20: note = AmDEL primer
  source 1..20: mol_type = other DNA
caagggttgcctggaagaac

SEQ ID NO: 12 - DNA, 20 bp, synthetic construct
  misc_feature 1..20: note = AmDEL primer
  source 1..20: mol_type = other DNA
ttgctcccagctcaactaca

SEQ ID NO: 13 - DNA, 17 bp, synthetic construct
  misc_feature 1..17: note = AmCHS primer
  source 1..17: mol_type = other DNA
gaggttcctcgtctcgg

SEQ ID NO: 14 - DNA, 19 bp, synthetic construct
  misc_feature 1..19: note = AmCHS primer
  source 1..19: mol_type = other DNA
caccgtaccaccagcaaag

SEQ ID NO: 15 - DNA, 19 bp, synthetic construct
  misc_feature 1..19: note = AmCH1 primer
  source 1..19: mol_type = other DNA
gatccaggggaagttcatc

SEQ ID NO: 16 - DNA, 19 bp, synthetic construct
  misc_feature 1..19: note = AmCh1 primer
  source 1..19: mol_type = other DNA
cgtcaatggcagtatcatg

SEQ ID NO: 17 - DNA, 19 bp, synthetic construct
  misc_feature 1..19: note = AmF3H primer
  source 1..19: mol_type = other DNA
gcttcgtttcgacatgtcg

SEQ ID NO: 18 - DNA, 24 bp, synthetic construct
  misc_feature 1..24: note = AmF3H primer
  source 1..24: mol_type = other DNA
agtttcatcaactcctcgctatac

SEQ ID NO: 19 - DNA, 19 bp, synthetic construct
  misc_feature 1..19: note = AmDFR primer
  source 1..19: mol_type = other DNA
ctgtttccaatttcggagg

SEQ ID NO: 20 - DNA, 20 bp, synthetic construct
  misc_feature 1..20: note = AmDFR primer
  source 1..20: mol_type = other DNA
tttgcaatgtctggaagcag

SEQ ID NO: 21 - DNA, 20 bp, synthetic construct
  misc_feature 1..20: note = PLT1 primer
  source 1..20: mol_type = other DNA
actttgtccatggggaccac

SEQ ID NO: 22 - DNA, 20 bp, synthetic construct
  misc_feature 1..20: note = PLT1 primer
  source 1..20: mol_type = other DNA
ccgtctcaacaacggctagt

SEQ ID NO: 23 - DNA, 20 bp, synthetic construct
  misc_feature 1..20: note = PLT2 primer
  source 1..20: mol_type = other DNA
agcaaccctggaatgcttca

SEQ ID NO: 24 - DNA, 20 bp, synthetic construct
  misc_feature 1..20: note = PLT2 primer
  source 1..20: mol_type = other DNA
ccgtggaaaactcatcccca

SEQ ID NO: 25 - DNA, 20 bp, synthetic construct
  misc_feature 1..20: note = CUC1 primer
  source 1..20: mol_type = other DNA
tgcatgagtatcgcctcgac

SEQ ID NO: 26 - DNA, 20 bp, synthetic construct
  misc_feature 1..20: note = CUC1 primer
  source 1..20: mol_type = other DNA
tcgtctgctctctaccgaca

SEQ ID NO: 27 - DNA, 20 bp, synthetic construct
  misc_feature 1..20: note = CUC2 primer
  source 1..20: mol_type = other DNA
cgcaaggttcttgacggttg

SEQ ID NO: 28 - DNA, 20 bp, synthetic construct
  misc_feature 1..20: note = CUC2 primer
  source 1..20: mol_type = other DNA
gtcggatacttccggtcacg

SEQ ID NO: 29 - DNA, 20 bp, synthetic construct
  misc_feature 1..20: note = STM primer
  source 1..20: mol_type = other DNA
cctccgtctcaagaagcgaa

SEQ ID NO: 30 - DNA, 20 bp, synthetic construct
  misc_feature 1..20: note = STM primer
  source 1..20: mol_type = other DNA
ggattgagtcctcctgcgag

SEQ ID NO: 31 - DNA, 20 bp, synthetic construct
  misc_feature 1..20: note = YUC4 primer
  source 1..20: mol_type = other DNA
tacacagcgtcttgcgtctt

SEQ ID NO: 32 - DNA, 20 bp, synthetic construct
  misc_feature 1..20: note = YUC4 primer
  source 1..20: mol_type = other DNA
gagtcttacgctgcccgatt

SEQ ID NO: 33 - DNA, 23 bp, synthetic construct
  misc_feature 1..23: note = TUB primer
  source 1..23: mol_type = other DNA
atgtttaggagggtgagtgagca

SEQ ID NO: 34 - DNA, 23 bp, synthetic construct
  misc_feature 1..23: note = TUB primer
  source 1..23: mol_type = other DNA
ctctcggcttctgtaaactccat

SEQ ID NO: 35 - DNA, 18 bp, synthetic construct
  misc_feature 1..18: note = GFP primer
  source 1..18: mol_type = other DNA
acaagttcagcgtgtccg

SEQ ID NO: 36 - DNA, 18 bp, synthetic construct
  misc_feature 1..18: note = GFP primer
  source 1..18: mol_type = other DNA
tcaccttgatgccgttct

(19) United States
(12) Patent Application Publication
(10) Pub. No.: US 2025/0154522 A1
(43) Pub. Date: May 15, 2025

(54) IMPROVED GENETIC TRANSFORMATION BY IMPROVING DE-NOVO SHOOT REGENERATION IN ADULT PLANTS AND DURING IN-VITRO TISSUE CULTURE

(71) Applicant: UNIVERSITY OF FLORIDA RESEARCH FOUNDATION, INCORPORATED, Gainesville, FL (US)

(72) Inventors: HEQIANG HUO, Gainesville, FL (US); ZHAOYUAN LIAN, Gainesville, FL (US)

(21) Appl. No.: 18/706,881

(22) PCT Filed: Nov. 2, 2022
(86) PCT No.: PCT/US2022/079184

§ 371 Date: May 2, 2024

Related U.S. Application Data
(60) Provisional application No. 63/274,714, filed on Nov. 2, 2021.

(51) Int. Cl. C12N 15/82

(52) U.S. Cl. CPC C12N 15/8262; C12N 15/8205

(57) ABSTRACT

Disclosed is an innovative method for genetic transformation through injection of Agrobacterium containing plasmid DNA expressing PLETHORAs (PLT5) or/and WOUNDINDUCED DEDIFFERENTIATION 1 (WIND1) in multiple plant species including Snapdragon and tomato. In addition, this plasmid has also been tested that it can significantly improve the genetic transformation method is not genotype dependent. Genetic transformation can be achieved by promoting de-novo regeneration of shoots in adult plants, which will of significant impact on some perennial plant species such as citrus and blue berry etc. The transgenic de-novo shoot regenerated from adult plants can by-pass the juvenile phase of plants regenerated through the tissue culture method. Expression of PLT5, WIND1, or both can also greatly improve plant regeneration and transgenic transformation through in-vitro tissue culture in recalcitrant plant species.

### STATEMENT OF GOVERNMENT SUPPORT

This invention was made with government support under Grant Nos. 2018-70016-27412 and 2022-70029-38471 awarded by United States Department of Agriculture, National Institute of Food and Agriculture. The government has certain rights in the invention.

### BACKGROUND

With the advent of genome editing and advancement of genomic sequencing, genetic transformation is becoming one of the most important biotechnology tools for functional study and modern plant breeding. However, success in genetic transformation is limited to the well-studied plant species, largely due to the low efficiency of the exogenous DNA delivery into plant cells and subsequent regeneration of plantlets (Altpeter et al. 2016). Exogenous DNA is typically delivered to plant cells through agrobacteria infection or biolistic bombardment, both of which require procedures for regenerating infected explants into plantlets in vitro (de Melo et al. 2020). Successful in-vitro plantlet regeneration through tissue culture is highly dependent on genotype and complicated by the application of hormone combination, thus resulting in success in limited plant species (Altpeter et al. 2016; Ikeuchi et al. 2019). Extensive attempts have been made to explore novel transformation methods to bypass the procedures of tissue culture-based transformation, yet little progress was achieved in most economically important crops despite that the floral dip method has been successfully applied to Arabidopsis and Setaria viridis (Martins et al. 2015; Saha and Blumwald 2016; Clough and Bent 1998). Recently, several studies showed that gene editing can be achieved by transient transformation of plant organs through plant viral vectors delivery system (Ellison et al. 2020; Ma et al. 2020; Ariga et al. 2020; Wang et al. 2020), but the small cargo capacities or/and narrow host range of these virus, and requirement of tissue culture for regenerating heritable edited plants limits their potential use to a few crop species (Wang et al. 2020; Dinesh-Kumar and Voytas 2020). In addition, transgenes delivered by viral vectors are temporarily expressed and unable to be inherited to next generation, thus this method cannot be applied for some function genomic studies or genetic engineering (e.g. gene overexpression). More recently, nanomaterials show promise as reagent delivery vehicles, however nanomaterial-mediated delivery is still less efficient than biotic delivery approaches and requires to be modified to increase its delivery efficiency (Nasti and Voytas 2021). Overall, the inability of transformation and inefficient plant regeneration are still the major barriers for plant biotechnology.

Plant cells exhibited remarkable developmental plasticity and totipotency, which lead to plant regeneration from diverse tissues in response to stimuli such as wounding and phytohormones (Gaillochet and Lohmann 2015). Extensive studies have reported on the effect of two categories of plant hormones auxin and cytokinin for developmental switch and organ regeneration. Generally, In vitro culture, a high ratio of auxin to cytokinin favors root regeneration, while a low ratio of auxin to cytokinin stimulates shoot regeneration, so the balance between auxin and cytokinin determines the fate of regenerated organs (Zhao et al. 2008). In addition, explants organogenesis in vitro consisted of two patterns: direct and indirect pathways. In indirect organogenesis, the primary explants is induced to form a disorganized and rapid proliferation mass of callus prior to shoot meristem formation; In direct organogenesis, there is no intervening callus proliferation stage (Brown and Thorpe 1986). Generally, a two-step tissue culture method is routinely used to induce shoot regeneration in plants, in which explants are first incubated on auxin rich callus-inducing medium (CIM) and subsequently on cytokinin-rich shoot-inducing medium (SIM) (Valvekens et al. 1988). Despite that this method has been proposed for several decades and adopted in limited genotypes of model plant species such as rice and tomato, efficient regeneration system has not been established in most crop species. Therefore, the recalcitrance to tissue culture and genetic transformation in many crops greatly restrain our ability to use genetic engineering and genome editing for precision breeding and functional genomics research. Extensive studies showed that the extraordinary pluripotency of plant somatic cells was governed by a complex regulatory network. Accumulating evidences from studies on model plant species Arabidopsis have provided better understanding of mechanisms underlying the plant regeneration through in vitro tissue culture. A range of developmental regulators such as such as WUSCHEL (WUS), PLETHORAs (PLTs), AUXIN RESPONSE FACTOR (ARFs), GROWTH-REGULATING FACTOR 4 (GRF4), LEAFY COTYLEDONS (LEC1 and LEC2), Baby-Boom (BBM), LATERAL ORGAN BOUNDARY DOMAIN (LBDs), CUP-SHAPED COTYLEDON (CUC1 and CUC2), CLAVAT3 (CLV3), SHOOT MERISTEMLESS (STM) and ENHANCED SHOOT REGENERATION (ESR) have been shown to determine cell fates to enhance callus formation or/and shoot regeneration (Ikeuchi et al. 2016; Ikeuchi et al. 2019).

In addition to hormone-induced de-novo shoot regeneration, wounding is another primary trigger for tissue repair and organ regeneration. Wounding induces expression of the AP2/ERF transcription factors WOUND INDUCED DEDIFFERENTIATION 1(WIND1) to promote cell dedifferentiation and proliferation for callus formation at the wound sites (Ikeuchi et al. 2017; Iwase et al. 2011). Overexpressing WIND1 and its homologous genes (WIND2-4) develops callus in the absence of exogenous hormones (Ikeuchi et al. 2017; Iwase et al. 2011). A recent study showed that WIND1 directly up-regulates the expression of ESR1 to promote CUC1-mediated shoot regeneration (Iwase et al. 2017). Iwase et al. (2013; 2015) also showed that the WIND-dependent regeneration pathway is conserved across diverse plant species, and ectopic expression of Arabidopsis WIND1 promoted callus formation as well as shoot regeneration in rapeseed (Brassica napus), tobacco (Nicotiana tabacum), and tomato (Solanum lycopersicum). Interestingly, the AP2/ERF transcription factors PLT3, PLT5 and PLT7 were also responsive to wound signals. The PLT3/5/7 triple mutant exhibit significant defects in callus induction, demonstrating that these genes participate in callus formation at wound sites (Ikeuchi et al. 2017). More recently, a study has demonstrated that PLT3/5/7 play an important role in vascular repair and regeneration from aerial organs after mechanic injuries. The PLT3/5/7 are greatly induced by wounding signals at the injury sites, which subsequently upregulate CUC2 transcription by directly binding its promoter. Both PLT3/5/7 and CUC2 upregulate auxin biosynthesis gene YUCCA4 (YUC4) to control local auxin production, which is essential for vascular regeneration (Radhakrishnan et al. 2020).

Recent years, extensive efforts were given to utilize some developmental regulators to improve the efficiency of plant regeneration and genetic transformation through tissue culture (Lotan et al. 1998; Boutilier et al. 2002; Zuo et al. 2002; Nelson-Vasilchik et al. 2018; Debernardi et al. 2020; Lowe et al. 2016; Maher et al. 2020). For example, Lowe et al. overexpressed the maize (Zea mays) Baby-Boom (BBM) and maize WUSCHEL2 (WUS2) genes, which produced high transformation frequencies in previously non-transformable maize inbred lines (Lowe et al. 2016). More recently, Debernardi et al (2020) improved the transformation efficiency of rice, wheat, and citrus by expressing a GRF-GIF chimeric protein. However, these methods require tedious and complex tissue culture procedures. In 2020, Maher et al. demonstrated that transgenic transformation and gene editing can be achieved by injection of mixtures of agrobacteria expressing WUS2, STM, and IPT of maize (Zea mays) in tobacco shoots (N. benthamiana) (Maher et al. 2020), but the successful transformation through this injection method was by far limited to tobacco.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 are schematic diagrams for the control expression vector with no DRs, POX135 (a) and DR-containing vectors (b). The control expression vector POX135 contained a cGFP-NPTII fused protein gene driven by a CsVMV (Cassava Vein Mosaic Virus) promoter and an anthocyanin production regulator DELIA (DEL) from A. majus under 2×CaMV35S promoter; the DRs-containing expression vectors contain different DRs (PLT5, ESR1, WUS-2A-BBM, WUS and WIND1) from Arabidopsis driven by a CaMV35s promoter (b). For WUS-2A-BBM, a short self-cleaving peptide (GSGATNFSLLKQAGDVEENPGP) from porcine teschovirus was included between WUS and BBM.

FIG. 2a-j shows that De novo shoot regeneration was promoted by Arabidopsis PLT5 in A. majus. (a) Primary and axillary shoots were cut off from the approximate 70-day-old snapdragon when the flower bud differentiation initiated, but a roughly 0.3 mm stem base of axillary shoots was remained for injection; (b) injection of the A. tumefaciens (GV3101) containing different DRs at the wound sites indicated by white arrows; (c) embryonic-callus-like tissues developed form the wound sites 7 days after injection indicated by the red arrow head; (d) De novo shoots (red arrow head) regenerated at wound sites 14 days after injections, while the shoot indicated by the white arrows initiated from the original axillary shoot meristems; (c) The red pigment due to the enriched anthocyanin in the stems of multiple regenerated shoots 25 days after injection; a red-purple stem was indicated by the white arrow head; (f) the representative image of a control plant with regenerated shoot (red arrow) from the original axillary after injection of GV3101 containing plasmid with no DR. (h) the representative image of a plant with the regenerated shoot (red arrow) after injection of GV3101 containing PLT5-plasmid; (g) and (j) the close-up images of shoots indicated by red arrows in (f) and (h), the white arrow in (g) indicating no shoots initiated from wounding sites.

FIG. 3a-b shows the effect of different DRs on the snapdragon shoot regeneration and transformation efficiency when delivered into the wound sites with GV3101 (a) or EHA105 (b). For each treatment, eighty sites were injected for multiple times. Data was collected, when the new shoots fully developed at ˜6 weeks after injection.

FIG. 4a-e shows transformation by stable GFP signals detection and PCR genotyping. (a) and (c) Detection of GFP fluorescence in the newly emerged leaves (a) or flower bud (c) of transgenic regenerated shoots; (b) and (d) Detection of GFP fluorescence in the newly emerged leaves (b) or flower bud (d) of non-transgenic shoots from the same plant; Red arrow indicated the leaves or flower bud that was used for GFP detection; (e) A PCR analysis to detect the GFP coding region P: Vector plasmid as a positive control; WT: Negative control, genomic DNA of non-transformed wild-type plant.

FIG. 5a-f shows detection of GFP fluorescent in the radicles of T1 transformed snapdragon seeds and roots. (a) and (b) The T1 seeds in the light field and dark field under UV light; (c) and (d) The root of non-transgenic plants in the light field and dark field under UV light, (e) and (f) The roots of transgenic plants in the light field and dark field under UV light.

FIG. 6a-b shows stable inheritance of transgenes to snapdragon T1/F2 progenies. (a) Segregation of the red-stem trait in T1/F2 seedlings; (b) PCR genotyping for GFP in five red-stem and green-stem T1/F2 seedlings, P: positive control, WT: wild type

FIG. 7a-c shows enriched anthocyanin in Tl transformed snapdragon due to the upregulation of anthocyanin biosynthetic genes. (a) images showing the color difference of the anthocyanin extracts from stems of wild type snapdragon plant (WT) or DEL-transformed plant (T1-R); (b) anthocyanin content determined by spectrophotometer; Data shown in (b) are mean values (n=5) with standard deviations; (c) relative expression of DEL and other anthocyanin biosynthetic genes. Average value for each transcript were calculated from 3 technical replication for per sample and three biological replications were applied to each sample. AmUBQ gene was used as internal reference control, stems samples were collected 40-days-old seedlings.

FIG. 8a-c shows shoot regeneration and genetic transformation of the soil-grown tomatoes (Solanum lycopersicum) through injection of A. tumefaciens (GV3101) with plasmid DNA containing PLT5. (a) Diagram to show two type of injection sites: the wounding surface on the top of shoots indicated by the white arrow head, and the axillary shoot meristems indicated by the white arrow; (b) the morphology of shoots regenerated from the wounding sites; (c) the morphology of shoots regenerated from the axillary shoot meristems

FIG. 9a-k shows tomato shoots regeneration promoted by PLT5 from wound sites. (a) single green shoot regenerated from axillary shoot meristems after injection of GV3101 containing plasmid DNA without DRs. (b) the close-up image of regenerated shoot from (a) to show no anthocyanin accumulation in its stem; (c) no callus-like tissue formed and shoot regenerated at the wound site after injection of GV3101 containing plasmid DNA without DRs. (d-k) multiple shoots initiated from the wound site with improved anthocyanin accumulation after injection of GV3101 containing plasmid DNA with PLT5. The indicated section by red arrow heads in d and f were magnified in e and g, respectively. (h-k) the two shoots initiated from the wound sites after injection with an obvious callus-like structure; (j) and (k) magnified details for the indicated section by red arrowhead in h and j. The callus-like structures are highlighted by arrowheads (k).

FIG. 10a-j shows transformation confirmation of regenerated shoots from wound sites in tomato. GFP fluorescence was detected in the transgenic regenerated tomato shoots (a-c) and the callus-like tissues from wound sites (a, d and e), but not in the control (FIG. 11 a and b). The enhanced anthocyanin derived from overexpression of snapdragon DEL were also observed in the stem (f), abaxial side of leaves (g) and flower sepals (h) but not in the control non-transgenic shoots (FIG. 11 c-d), b and d were magnified details from the overview of the regenerated shoot (a), indicated by the white arrowheads. (j) PCR genotype results indicate the presence of transgene in these regenerated shoots from wound sites.

FIG. 11a-e shows the counterpart control of regenerated tomato shoots after injection in the FIG. 10, a and b No GFP signal was detected in the leaves of regenerated shoots in the control group. c-e No obvious anthocyanin accumulated in the stems, abaxial side of leaves and flower sepals of regenerated shoots in the control group.

FIG. 12a-f shows the callus-like structure could be induced by injecting agrobacterium solution containing PLT5 in Bok Choy. (a) An image to show the injection site for a control plasmid indicated by the white arrow, (b-c) Close-up images of (a) to show only dead tissue formation at the wound site of stems after injection with DR-free plasmids; (d) An image to show the injection site indicated by the white arrow for PLT5 plasmid; (c-f) Close-up images of (d) to show live callus-like tissue formation at the wound site of stems after injection with PLT5 plasmid; the callus-like tissues were indicated by the red arrow heads. Images in b and e were taken at 14 days after injection; Images in c and f were taken at 21 days after injection.

FIG. 13a-e shows Induction of the embryo-like structure formation by PLT5 at wound site in soil-grown sweet pepper. (a) An image to show the injection site, (b and d) The embryo-like structures formation due to PLT5 overexpression at the wound sites, (c and c) Magnified details of embryo-like structures at wound sites in b and d, respectively.

FIG. 14a-e shows the regeneration and transformation of Bok Choy by using cotyledon as the explant on the MS phytohormone-free medium. a. The root was regenerated from the petiole of cotyledon when over-expressed PLT5 after 10 days culture, but not in the control treatment group in b; (c). the callus formed from the newly regenerated roots; and the GFP could be detected in the roots and callus after 3 weeks culture (d and c). The bar=3 mm.

FIG. 15a-h shows Successful plant regeneration from roots tips transformed with PLT5 plasmid in Bok choy. (a) Newly regenerated root from callus derived from the culture of cotyledons on phytohormone-free MS media, as shown in the FIG. 14. (b) Magnified image of root tip in the light field; (c) GFP fluorescence detected in the root; (d) Detached root tips for further culture on the media with 3 mg/L 6-BA; (E) the green callus was formed after 1 month of culture on the MS media with 3 mg/L 6-BA; (f) and (g) the embryo and shoots were developed from green calluses; and (h) the GFP fluorescence detected in the regenerated shoots.

FIG. 16a-d shows that PLT5 promotes shoot regeneration from embryogenic callus of Bok Choy. (a) callus formation from cotyledon explants after infections with PLT5-free plasmid. (b) formation of embryogenic callus from cotyledon explants infected with plasmid containing PLT5; (c) formation of shoot meristem with strong GFP signal from embryogenic callus; (d) regeneration of shoots with strong GFP signal from shoot meristems due to expression of PLT5. All cotyledon explants were cultured on the MS medium with 3 mg/L 6-BA only directly. Pictures were taken from the same explant at different developmental stages.

FIG. 17a-h shows ectopic expression of PLT5 improved Bok choy regeneration and transformation efficiency through inducing embryogenesis pathway in a short time. (a) green embryogenic tissue initiated from white calluses after 14 days of culture on the media containing 3 mg/L 6-BA. The embryo (c-d) and shoots (f-g) developed after another one week of culture. (b, c, h) strong GFP fluorescence detected in these green tissues from (a), somatic embryos from (d), shoots from (g); d and g were magnified images of c and f respectively. The embryo-like structures are indicated in c by a red arrow.

FIG. 18a-c shows relative expression of key regulators for shoot regeneration in calluses of Bok Choy. (a) The control callus sample with GFP fluorescence; (b) The green callus tissues from infection with PLT5 plasmid; (c) Relative expression level of regulatory genes that are downstream of PLT5 in Bok Choy calluses. Average value for each transcript were calculated from 5 biological replications and 3 technical replications for each sample. TUB gene was used as an internal reference control, all callus samples were collected after 2 weeks culture after Agrobacteria infection. Scale bars=3 mm.

FIG. 19a-k shows conformation of the Bok Choy regeneration through embryogenesis induced by ectopic expression PLT5. (a) the regenerated embryos after infection, which were magnified in (b), Images of Bok Choy seeds (c), regenerated callus from explants infected with the DR-free plasmid (d), embryogenic callus/somatic embryos from explants infected with the PLT5 plasmid (e) and wildtype seedling (f) before staining with Sudan Red 7B. (g-k) The respective images of counterparts after Sudan Red 7B staining. Scale bars: 2 mm

FIG. 20a-h shows the effect of PLT5 on shoot regeneration and genetic transformation in Pei-Tsai. (a-b) The callus formed from Pei-Tsai explants infected with DR-free plasmid; (c) no GFP fluorescence detected in the callus from (b). (d) The embryos induced by PLT5; (e) magnified embryos and GFP fluorescence detected in the same embryos from (d); (f) an image of somatic embryos from (d) at a later developmental stage and GFP detected in the same embryos; (g) an image of shoots regenerated from (f) and GFP fluorescence detected in the regenerated shoots. The bar=5 mm.

FIG. 21a-h shows transformation confirmation in Bok Choy and Pei-Tsai. (a) Rooting of the regenerated Bok Choy seedling on the phytohormone-free MS medium; GFP was detected in the stems of transformed seedlings of Bok Choy (e-g); but not in the stem of wildtype control plants (b-d); (h) PCR genotyping for GFP presence in the transformed Pei-Tsai lines (Pt) and Bok Choy (Bc) The bar=5 mm.

FIG. 22a-j shows ectopic expression of PLT5 improve sweet pepper transformation efficiency through tissue culture. a-c No transformed shoots regenerated from callus, which was generated form cotyledon, although the ectopic anthocyanin accumulation was detected indicated by the red arrow head in the c. d The leave-like structures were regenerated directly from the hypocotyl bypass the callus stage, but no obvious anthocyanin was found in the leave, the detail was magnified in the e. f and g The embryo-like structure indicated by the white arrow head was induced by ectopic expression of PLT5 in the callus which was cut off from the cotyledon after 2 weeks culture, the embryo-like structure developed into leaf-like structure after one more month of culture shown in h, the anthocyanin accumulation on the leave was found as shown in the j.

FIG. 23a-e shows the comparison of anthocyanin accumulation in the callus and leaves of shoots initiated from the hypocotyl without callus formation (a-b) and regenerated shoots initiated from the cotyledon calluses with the aid of PLT5 (c-e). The anthocyanin accumulation in the calluses were indicated by white arrows. The bar=5 mm

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “Agrobacterium” or plural form “Agrobacteria” refers to a genus of Gram-negative bacteria that uses horizontal gene transfer to cause tumors in plants. The ability of Agrobacterium to transfer genes to plants and fungi is used in biotechnology, in particular, genetic engineering for plant improvement.

The terms “clone”, “cloning”, or “cloned” as used herein, unless otherwise indicated, refers to the process of making multiple molecules. Molecular cloning is a set of experimental methods known in the art of molecular biology that are used to assemble recombinant DNA and protein molecules and to direct their replication or expression within host organisms.

The term “cloning site” refers to a short segment of DNA which contains restriction sites. The term “multiple cloning site” refers to a short segment of DNA which contains many (up to ˜20) restriction sites. The purpose of a multiple cloning site in a plasmid is to allow a piece of DNA to be inserted into that region.

“Expression” refers to the production of a functional product. For example, expression of a nucleic acid fragment may refer to transcription of the nucleic acid fragment (e.g., transcription resulting in mRNA or functional RNA such as RNAi or ribozymes) and/or translation of mRNA into a precursor or mature protein.

The term “gene” includes cDNAs, RNA, or other polynucleotides that encode gene products.

“Heterologous” with respect to sequence means a sequence that originates from a foreign species, or, if from the same species, is substantially modified from its native form in composition and/or genomic locus by deliberate human intervention.

“Introduced” or “introducing”, or other grammatical forms thereof, in the context of delivering a nucleic acid fragment (e.g., a recombinant DNA construct) into a cell, means “transfection”, “transformation”, or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The terms “nucleic acid.” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “sequence” refers to a nucleotide sequence of any length, which can be DNA or RNA; can be linear, circular or branched and can be either single-stranded or double stranded.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment. The term “plant” includes reference to whole plants, plant organs (e.g., leaves, stems, roots, etc.), seeds and plant cells and progeny of same. “Plant cell,” as used herein, includes, without limitation, cells in or from seeds, suspension cultures, embryos, meristematic regions, callus tissue, leaves, roots, shoots, gametophytes, sporophytes, pollen, and microspores. The class of plants which can be used in the methods of the invention is generally as broad as the class of higher plants amenable to transformation techniques, including both monocotyledonous and dicotyledonous plants. The term “adult plant” refers to a fully mature plant that is able to reproduce.

The term “PLETHORA” or “PLT” “refers to the AP2/ERF transcription factor that have an important role in callus formation and organ regeneration during in-vitro tissue culture and during wound repairing after mechanic injuries of aerial organs. PLETHORA factors include PLT3, PLT5 and PLT7. While a PLT gene from Arabidopsis is exemplified herein, PLT3, PLT5, and/or PLT7 genes may be implemented as well. In addition, PLT3, PLT5 and/or PLT 7 homologs from other plant species may be implemented as well.

An “expression cassette” is generally understood in the art as a nucleic acid construct (e.g. Recombinant DNA construct) that contains a transgene and the necessary regulatory sequences to enable expression of the transgene.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

The term “recalcitrant plants” refers to plants that are difficult to be regenerated and/or transformed through different measures including Agrobacterium-mediated and Biolistic bombardment etc. These plants include but not limited to most varieties of field crops such as corn, wheat, sorghum, cotton, soybean, canola, sunflower etc., most vegetable species such as B. rapa cabbages (e.g. bok choy), cauliflower, broccoli, spinach, squash, melons, cucumber, carrots etc. most fruit trees such as blue berry, citrus, black berry, grapes, peach, plum, pear, apple, sweet pepper, ornamentals such as snapdragon, rose, poinsettias, dogwood etc. and forest trees such as poplar, willow, pine etc.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. “Genome” as it applies to plant cells encompasses not only chromosomal DNA found within the nucleus, but organelle DNA found within subcellular components (e.g., mitochondrial, plastid) of the cell.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Somatic embryogenesis (SE)” is herein defined as the developmental process by which somatic cells undergo restructuring to generate embryogenic cells. These cells then go through a series of morphological and biochemical changes that result in the formation of a somatic or non-zygotic embryo capable of regenerating plants. Somatic embryogenesis represents a unique developmental pathway that includes a number of characteristic events: dedifferentiation of cells, activation of cell division, and reprogramming of their physiology, metabolism, and gene expression patterns.

Somatic embryos can differentiate either directly from the explant without an intervening callus phase or indirectly after a callus phase, referred to as direct somatic embryogenesis (DSE) and indirect somatic embryogenesis (ISE), respectively (Sharp et al., 1980 Hortic. Rev. 2:268-310).

Embryogenic cultures capable of plant regeneration are essential for transformation by particle bombardment or Agrobacterium mediated transformation. Regeneration of mature plants via somatic embryogenesis can be divided into three stages: (1) primary induction of somatic embryos, (2) establishment of proliferative embryogenic cultures, and (3) generation of whole plants by embryo maturation and germination. Genetic variation in primary embryogenesis has been observed and studied extensively (Kita Y. et al (2007) Plant Cell Rep 26:439-447).

The lack of knowledge of the factors controlling somatic embryogenesis, the asynchrony of somatic embryo development, and low true-to-type embryonic efficiency are some of the factors that limit the application of somatic embryogenesis for regeneration of transformed plants from tissue culture.

“Regeneration,” or “regenerating” or other grammatical forms thereof, as used herein, refers to a morphogenetic response that results in the production of new tissues, embryos, organs, whole plants or fragments of whole plants that are derived from a single cell, or a group of cells. In the present invention, the term “regeneration” encompasses production of new tissues, organs, whole plants or fragments of whole plants that are derived from a single cell, or a group of cells. Regeneration may proceed indirectly via somatic embryogenesis or directly without an intervening somatic embryo formation phase.

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

The terms “stably integrating” or stable integration” refers to the permanent expression of the gene of interest into the nuclear genome. A stable transfection allows foreign DNA to be fully integrated into the host genome, therefore allowing indefinite expression of the foreign DNA.

“Selectable marker” provides a means of selecting the desired plant cells. Vectors for plastid transformation typically contain a construct which provides for expression of a selectable marker gene. Marker genes are plant-expressible DNA sequences which express a polypeptide which resists a natural inhibition by, attenuates, or inactivates a selective substance, i.e., antibiotic or herbicide. Alternatively, a selectable marker (reporter) gene may provide some other visibly reactive response, i.e., may cause a distinctive appearance or growth pattern relative to plants or plant cells not expressing the selectable marker gene in the presence of some substance, either as applied directly to the plant or plant cells or as present in the plant or plant cell growth media. These visual selectable marker genes are called “reporter genes.” Reporter genes include GUS and GFP.

The term “transgene” refers to a nucleic acid comprising a coding sequence or a transcribable DNA sequence that is transfected into a cell, and may optionally be operably linked to requisite regulatory sequences to induce or control transcription and/or translation.

The terms “WIND1” or “WOUND INDUCED DEDIFFERENTIATION 1” refers to the AP2/ERF transcription factor that promotes cell dedifferentiation and proliferation for callus formation and organ regeneration at the wound sites of in-vitro explants and the wound sites of arterial organs. While WIND1 from Arabidopsis is exemplified herein, WIND1 homologs from other plant species can be implemented as well.

The term “vector” as used herein refers to a nucleic acid capable of transporting another nucleic acid to which it has been linked to a plant cell. A vector may be a recombinant DNA construct. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### DETAILED DESCRIPTION

The present disclosure is based on studies involving the utilization of two wound-induced transcription factors WIND1 and PLT5, and three developmental regulators, ESR1, BBM and WUS for de-novo shoot regeneration after mechanical injuries and in-vitro shoot regeneration through tissue culture. Disclosed is an innovative method for genetic transformation involving Agrobacteria containing plasmid DNA engineered to express PLETHORAs (PLT5) or/and WOUNDINDUCED DEDIFFERENTIATION 1 (WIND1). The methods taught herein have been successful in transforming multiple plant species including Snapdragon and tomato. Genetic transformation can be achieved by promoting de-novo regeneration of shoots in adult plants. The transgenic de-novo shoot regenerated from adult plants can by-pass the juvenile phase of plants regenerated through other tissue culture methods. Expression of PLT5, WIND1. or both has also been demonstrated to improve plant regeneration and transgenic transformation through in-vitro tissue culture in recalcitrant plant species.

### Overview

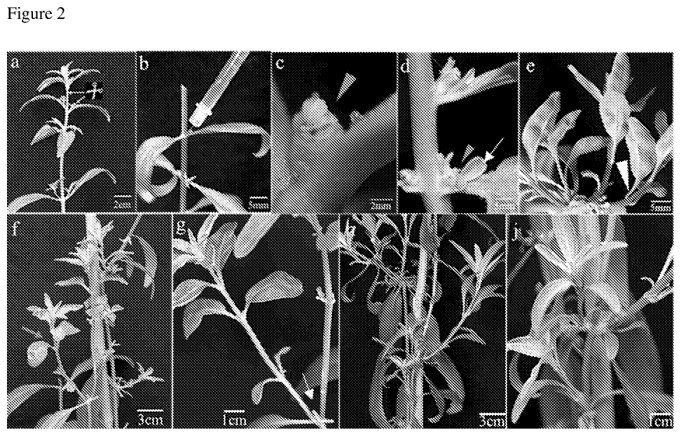

Plant regeneration and genetic transformation are fundamental and essential for genetic engineering of plants. Until today, only a very limited number of plant species are amenable to be transformed, and successful transformation is highly genotype dependent. Achieving stable transformation of plants remains a major hurdle except in a few species, hindering efficient progress towards both basic and applied scientific goals. In recent years, developmental regulators have been utilized for improving plant regeneration and genetic transformation through tissue culture and injection of agrobacterium to aboveground meristems (Maher et al. 2020; Lowe et al. 2016). The effect of several developmental regulators were examined in promoting plant regeneration and genetic transformation. Among the tested regulators, WUS and BBM that has been extensively used for monocot species (Lowe et al. 2016; Jones et al. 2019; Hoerster et al. 2020); however, there has been limited success using WUS and BBM in dicotyledonous species (Heidmann et al. 2011; Zhang et al. 2021). The experimental results disclosed herein show that WUS exhibited effects in promoting snapdragon and tomato shoot regeneration and genetic transformation through agrobacterial injection, but the efficiency is much lower compared to the transformation event promoted by PLT5 (Table 1 and 2). A very recent report showed that PLT5 was a master regulator for stem vascular repair after wounding in Arabidopsis (Radhakrishnan et al. 2020). Wound signals induce and enhance transcription of PLT5 at wound sites, leading callus formation at first and subsequent differentiation into vascular tissues (Radhakrishnan et al. 2020). In consistent with this result, it was also observed that callus-like tissues were first formed at injection sites and regeneration of multiple shoots from these callus-like tissues (FIG. 2d-k). Interestingly, all transformed shoots were derived from these callus-like tissues; while none of the regenerated shoots from the Agrobacterium-infected axillary meristems were transgenic (Table 2). It is postulated that dedifferentiation reflected by callus formation and re-differentiation reflected by de-novo shoot regeneration from these calluses at the wound sites are critical for the success of gene delivery into plant cells with the help of PLT5. This progress highly resembles the genetic transformation through in-vitro tissue culture generally consisting of a procedure with a higher concentration of auxin for callus formation and subsequent culture with a higher cytokinin for shoot regeneration. Indeed, the transformation efficiencies in many plant species such as snapdragon (Lian et al. 2020), trifoliate orange (Poncirus trifoliata L. Raf.) (Zou et al. 2008) and common bean (Phaseolus vulgaris L.) (Mukeshimana et al. 2013; Collado et al. 2016) were much higher when plant regeneration acts through an indirect organogenesis pathway that requires the formation of callus or somatic embryo. This may be explained by the timing window for Agrobacteria infection being larger.

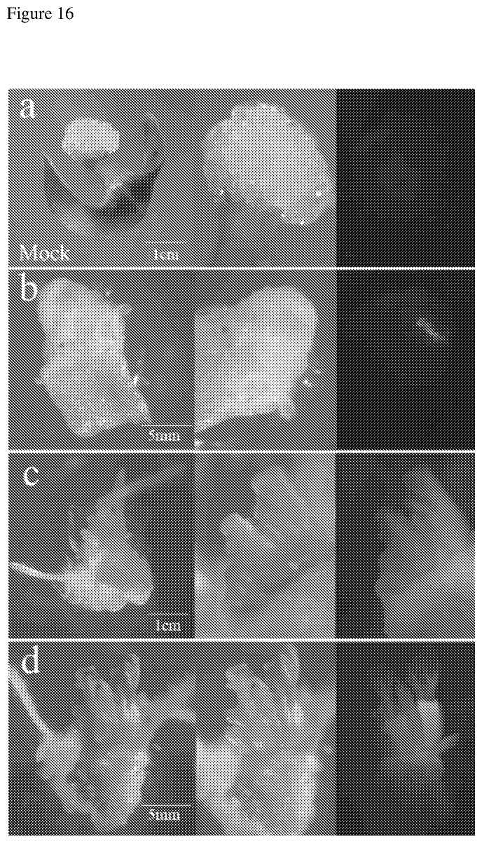

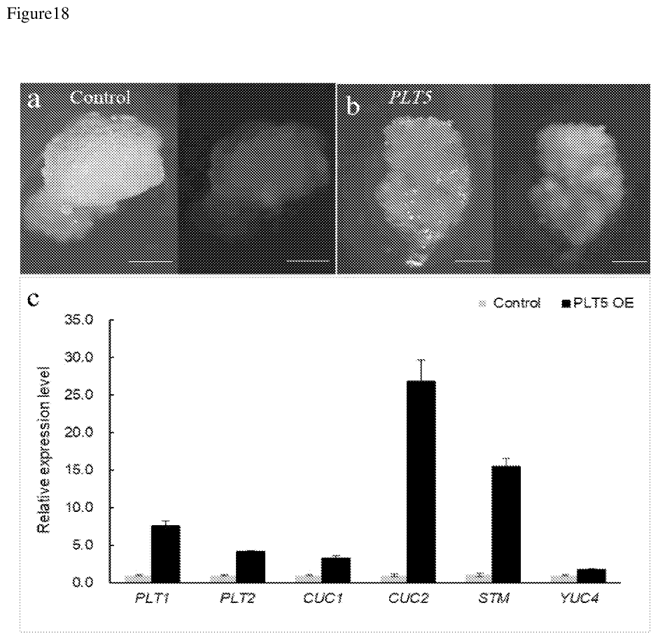

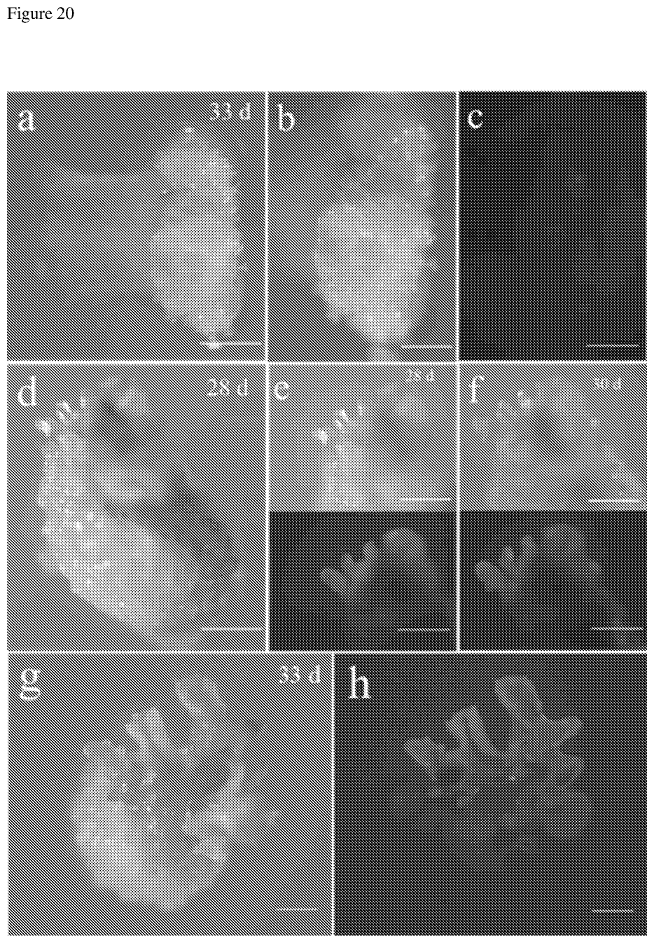

In addition, data is disclosed herein demonstrating that PLT5 can also promote plant regeneration and genetic transformation through in-vitro tissue culture. PLT5 is shown to respond to wound signals, promotecallus formation from in-vitro explants and acquire cell pluripotency for de-novo shoot regeneration (Shin et al. 2020), which is believed to be extremely important for inducing cell dedifferentiation to form callus to regain cell pluripotency in the adult plant injection, since plant cell regeneration ability gradually decreased with age (Ye et al. 2020). These characteristics are also important for some specific plant species or specie varieties with low regeneration ability in tissue culture. The results provided herein show that PLT5 appears essential in acquiring cell pluripotency in sweet pepper and two B. rapa varieties since no shoot regenerated from the callus without the aid of PLT5 (FIGS. 16 and 20). It was reported that PLT3/5/7 control plant regeneration via a two-step mechanism, where the root stem cell regulators PLT1/2 are first activated for establishing pluripotency and CUC2 gene is required for completion of shoot regeneration (Kareem et al. 2015). qPCR results disclosed herein show that the CUC2 and PLT1/2 are substantially increased in the PLT5-expressing tissues, while the increase of YUC4 transcript is not remarkable (FIG. 18). Mathew (2021) et al also found that PLT3/5/7 require YUC4 and CUC2 but not PLT1/2 for vascular regeneration in damaged aerial organs (Mathew and Prasad 2021). This indicates that the mechanism for shoot regeneration from in-vitro cultured explants is different from the organ regeneration from the aerial damaged tissues. However, the data presented herein shows that PLT3/5/7 and CUC2 appear essential for both types of organ regeneration, which could be explained by the response of PLT3/5/7 to wound signal in both scenarios (Kareem et al. 2015; Mathew and Prasad 2021).

WIND1 is parallel to PLT3/5/7, which responds to wound signals to promote callus formation at wound sites of in-vitro explants (Iwase et al. 2011). The AP2/ERF transcription factor WIND1 controls cell dedifferentiation in Arabidopsis. (Iwase et al. 2011). WIND1 promote callus formation and shoot regeneration through direct transcriptional activation of ESR1 at wound sites of in-vitro explants (Iwase et al. 2017). Application of ESR1 did not promote shoot regeneration from wound sites of aerial organs as WIND1 did, which can be attributed to the difference in molecular regulation for de-novo shoot regeneration from in-vitro explants and that for shoot regeneration from aerial damaged organs, as observed for PLT3/5/7 (Kareem et al. 2015; Mathew and Prasad 2021). Previously, WIND1 was strongly upregulated in the vasculature and epidermis of the scion's hypocotyl, despite that reducing the ability to form wound-induced callus in Arabidopsis by suppressing WIND1 targets had no effect on phloem connection (Melnyk et al. 2015). It will be noteworthy to elucidate whether co-expression of PLTs and WIND1 will have synergistic effect in promoting shoot regeneration and genetic transformation through tissue culture and injection of agrobacterium into wound organs.

Because of the small size, the Delila gene from A. majus was used for transformation selection. Delila was a well-known bHLH transcription factor that involves in the anthocyanin biosynthesis through MYB-bHLH-WD repeat (MBW) transcriptional complex model (Lloyd et al. 2017; Schwinn et al. 2006). Heterologous expression Delila could enhance anthocyanin accumulation in Nicotiana tabacum and Lilium (Naing et al. 2017; Fatihah et al. 2019). However, enhanced anthocyanin production due to heterologous Delila expression was not found in two cabbages varieties. By contrast, ectopic expression of Delila in snapdragon, tomato and sweet pepper resulted in increases in anthocyanin in varying degrees, which suggested that Delila function might be species dependent.

### Construction and Elements of Vectors

The vector of the disclosure in certain embodiments comprises expression cassettes which comprises the necessary genetic elements to stably transform a target plant cell with a foreign (heterologous) DNA (e.g. gene of interest) coding for a molecule of interest, like a phenotype to be expressed by the plant or a non-plant high value molecule, like a biologically active peptide (or polypeptide). The vector is constructed with one or more sequences that code for transcription factors that improve genetic transformation and de-novo shoot regeneration. In certain embodiments, the transcription factors are in a separate expression cassette from the molecule of interest. In specific embodiments, the transcription factors that improve genetic transformation and de-novo shoot regeneration are WIND1 and/or PLT5. The vector may be constructed with one or both transcription factors, and the transcription factors are operably linked to a promoter region. In a specific embodiment, the promoter region is CaMV35S for facilitating high level of RNA transcription in a wide variety of plants. The vector is inserted into a transcriptionally active region in the plant genome.

In particular embodiments the vector or expression cassettes comprise at least one transgene comprising a coding sequence or a transcribable DNA sequence operably linked to a plant-expressible promoter. In certain embodiments the transgene comprises a gene of interest. In some embodiments the transgene comprises a protein coding sequence. In other embodiments the transgene comprises a transcribable DNA sequence encoding a non-coding RNA molecule (e.g. RNAi, ribozyme, and the like). In yet other embodiments the transgene comprises a marker gene.

In certain embodiments the marker gene is a selectable marker gene. In some embodiments the selectable marker gene comprises an adenylyltransferase (aadA) gene, a neomycin phosphotransferase (nptII) gene, a hygromycin phosphotransferase (hpt, hph or aph IV), 5-enolpyruvylshikimate-3-phosphate synthase (EPSPS) gene, a dicamba monooxygenase (DMO) gene, or a bialaphos resistance (bar) or phosphinothricin N-acetyltransferase (pat) gene. In another embodiment the selectable marker gene comprises an adenylyltransferase (aadA) gene. In additional embodiments the marker gene is a screenable marker gene. In various embodiments the screenable marker gene comprises a green fluorescent protein (GFP) or a β-glucuronidase (GUS) gene. In other examples, the selectable marker is an anthocyanin gene (e.g. DEL). While selectable markers can be used for selection, and can be placed with other cassette (PLT5, WUS1, WIND1, BBM-WUS) for selection, they are not essential or required for selections in recalcitrant species such as pepper and Bok Choy. Typically, without PLT5, WIND1, no seedlings can be regenerated from infected explants, which means that the regeneration itself can serve as a selection mechanism. As such, embodiments herein can provide selection-free vectors and constructs.

In one example, the expression cassette comprises, operably joined, a transcriptional initiation region functional in a plant genome, at least one heterologous DNA sequence coding for a target molecule of interest, e.g. a gene of interest (or functional fraction thereof) encoding a biologically active compound, and control sequences positioned upstream for the 5′ and downstream from the 3′ ends of at least one heterologous DNA and a transcription termination region to provide expression of the coding sequence in the genome of a target plant. Preferably, the expression cassette is flanked by plant DNA sequences, in order to facilitate stable integration of the expression vector into the plant genome. In the construction of the expression cassette, DNA sequences are included that comprise one or more cloning site(s) such as for integration of the gene(s) of interest.

As alluded to above, it will be generally advisable for certain research scenarios to have at least one additional heterologous nucleotide sequence coding for a selectable phenotype, such as a gene providing for antibiotic resistance or a functional portion thereof to serve as a selection marker associated with the expression cassette or with the universal integration expression vector. This facilitates identification of the cells stably transformed with the foreign gene. Selectable marker genes are known in the literature, for instance aminoglycoside phosphotransferase, kanamycin and neomycin resistant genes such as NPTII, the gene which encodes aminoglycoside phosphotransferase. In some embodiments, the selectable marker comprises GFP.

### Transformation Methods

The disclosure also provides transformation methods which can produce integration of foreign genes into the genome of the plant cell after a selection process. An example of a method for transforming a plant uses a vector constructed with transgenes encoding WIND1, PLT5, or both to promote genetic transformation and de-novo shoot regeneration. Any method of transformation of the plant may be used. It a specific method, Agrobacteria are used to mediate delivery of expression constructs that are integrated into a plant genome. Any gene (or functional portion thereof) which may be utilized to transform a plant genome and encode a desired peptide to confer the desired trait to the target plant is suitable for transformation with the vector.

In certain embodiments, the methods of transformation disclosed utilize agrobacterium to integrate a foreign gene(s) into the genome of the plant cells. In a specific embodiment, the agrobacterium is Agrobacterium tumefaciens. The agrobacteria are transformed with the vector and suspended in an induction media. In some embodiments, the agrobacteria are transformed using heat and thaw methods. In some embodiments, the induction media comprises calluses induction media, shoots induction media, roots induction media, or any combination thereof.

The disclosure further provides a method of injecting agrobacteria transformed with the vector a target plant. In some embodiment, the target plant is injected multiples at multiple injection sites. The injection sites maybe a wound site or stem bases of axillary shoots. After injection, the vector of the disclosure improves genetic transformation and de-novo shoot transformation.

Agrobacterium-mediated gene transfer exploits the natural ability of Agrobacterium tumefaciens and A. rhizogenes to transfer DNA into plant chromosomes. Agrobacterium is a plant pathogen that transfers a set of genes encoded in a region called T-DNA of its tumor-inducing (Ti) plasmid or hairy-root-inducing (Ri) plasmid into plant cells at wound sites. This process depends on the cis acting T-DNA border sequences that flank the transferred DNA and the trans acting virulence (vir) functions encoded by the Ti plasmid or Ri plasmid and the bacteria's chromosome. The typical result of gene transfer in A. tumefaciens is a tumorous growth called a crown gall. The result of gene transfer in A. rhizogenes is hairy root disease. In both cases, gene transfer results in stable integration of the T-DNA region into a plant host chromosome. The ability to cause crown gall disease or hairy root disease can be removed by deletion of the oncogenic genes in the T-DNA without loss of DNA transfer and integration.

When the oncogenic genes are removed in this manner, the Agrobacterium is said to be disarmed or non-oncogenic.

Such Agrobacterium-mediated gene transfer systems are modified to contain a heterologous or foreign nucleotide sequence of interest, such as a foreign gene or genes of interest, to be expressed in the transformed plant cells. The heterologous nucleotide sequence to be transferred is incorporated into the T-DNA region, which is flanked by imperfect 25-bp terminal repeats or T-DNA border sequences that define the end points of an integrated T-DNA. Any sequences between these terminal repeats become integrated into the plant nuclear DNA (Fraley et al. (1983) Proc. Natl. Acad. Sci. USA 80:4803; Watson et al. (1985) EMBO J. 4:277; Horsch et al. (1985) Science 227:1229; Hernalsteens et al. (1984) EMBO J. 3:3039; Comai et al. (1984) Nature 317:741; Petit et al. (1986) Mol. Gen. Genet. 202:388-393; Shah et al. (1986) Science 233:478; Bytebier et al. (1987) Proc. Natl. Acad. Sci. USA 84:5345; Schafew ct al. (1987) Nature 327:529; Mcknight et al. (1987) Plant Mol. Biol. 8:439-445; Potrykus (1990) Biotechnol 8:535; Grimsley et al. (1987) Nature 325:177; Gould et al. (1991) Plant Physiol. 95:426; Ishida et al. (1996) Nature Biotechnology 14:745; and U.S. Pat. No. 5,591,616, and the references cited therein).

As will be evident to one of skill in the art, any nucleic acid of interest can be used for transformation using the methods of the present invention. Any recombinant construct comprising a nucleic acid of interest that may be introduced into a plant cell may be employed in the methods of the present invention. A plant can be engineered to express genes conferring different phenotypes, examples of such genes include, but are not limited to, disease and insect resistance genes, genes conferring nutritional value, genes to confer male and/or female sterility, antifungal, antibacterial or antiviral genes, and the like. Likewise, the method can be used to transfer any nucleic acid to control gene expression. Examples of nucleic acids that could be used to control gene expression include, but are not limited to, antisense oligonucleotides, suppression DNA constructs, or nucleic acids encoding transcription factors.

Genes of interest can be genes conferring one or more modified agronomic traits and characteristics including, but not limited to, increased yield, increased heterosis, increased oil and increased nutritional value. General categories of genes of interest include, for example, those genes involved in information, such as Zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products.

### EXAMPLES

### Materials and Methods

Plant materials. Snapdragon (Antirrhinum majus) stock line “JI2” and “Sippe50” were kindly provided by John Innes Center, UK. Inbred hybrid snapdragon seeds were derived from 6th round selfing of “JI2×Sippe50”. Tomato “Big Beef” seedlings. The seeds of Bok Choy and Pai-tsai (long white stalk) and Sweet Pepper (California wonder) were purchased in the local plant market

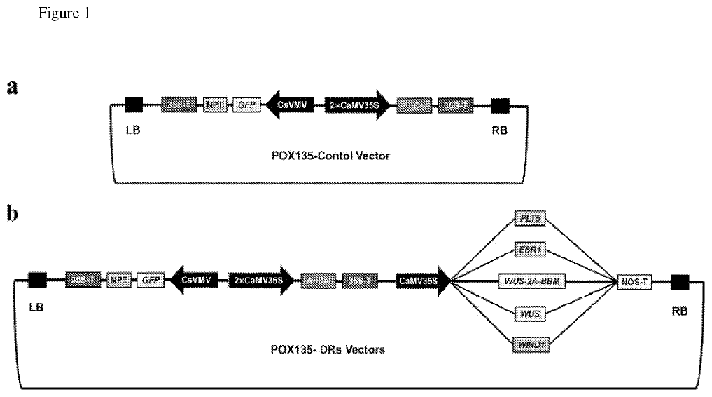

Vectors construction. A previous binary expression vector containing fused gene encoding Green Fluorescent Protein (GFP) with NPTII under a double-enhanced CsVMV (dCsVMV) for Kanamycin selection was modified by the following steps. The AtU6 promoter cassette of the PHN-SpCas9-4×Bsal-GFP was first removed through a digestion with AvrII; two oligos for omega enhancer with two Bsal cloning sites were synthesized, annealed and inserted into the plasmid at the AscI digestion sites to replace the SpCas9 gene; the 2×CaMV35S were amplified from the pGWB402 Omega and inserted into the construct through HindIII digestion and ligation to form our overexpression vector POX135 as shown in FIG. 1 (Nakagawa et al. 2007; Nguyen et al. 2021).

A well-known transcription factor Delila (Accession number: M84913.1) for anthocyanin biosynthesis in A. majus was synthesized and ligated into the POX135 at two Bsal cloning sites to form POX135-DEL (FIG. 1). In addition, two different plant DRs (Developmental Regulators) genes AtESRI (AT1G12980) and AtWIND1 (AT1G78080) were PCR cloned from Arabidopsis Columbia ecotype with primers AtESRAscIF3/AtESRBamHR3 and AtWIND1AscIF3/AtWIND1BamHR3, respectively (Table 6). Three genes of AtWUS (AT2G17950), AtPLT5 (AT5G57390), and a fused WUS-BBM gene linked with a LP4/2A linker (Sun et al. 2017), WUS-P2A-BBM (AT5G17430) were DNA synthesized. To make a short version cassette of CaMV35S::DRs::NOS-Terminator, the OCS terminator of PGSA1165 (www.arabidopsis.org) was replaced by the NOS terminator amplified with the primer sets NOSterSpeI-F and NOSterHindIII-R from pGWB402omega through digestions with Spel and HindIII and a subsequent ligation. Each DR was ligated to the resultant vector CaMV35S::NOS-Terminator through digestions with AscI and BamHI and a subsequent ligation. The CaMV35S::DRs::NOS were amplified with the primer set of 35S-AatII-F/NOSterMluI-R and ligated to POX135-DEL at the AatII and MluI restriction sites and to form the final expression plasmids with no or with different DRs (FIG. 1). All plasmid DNAs were transformed into A. tumefaciens strain EHA105 or GV3101 through heat and thaw methods.

Transformation through injection of agrobacterium with POX135-DEL. The seeds of plants were first germinated in a Petri dish with a moistened blot paper; germinated seeds were transferred to Pro-Mix soil, and grown in the growth chamber with 16 hours light/8 hours dark photoperiod at 28° C. The A. tumefaciens was first culture in the liquid LB (LB Broth, Powder (Miller's), Thermo Scientific) overnight at 28° C., and agrobacteria cells were collected by centrifugation at 4500 rpm for 15 mins and resuspended to an O.D.600 of 0.8 with liquid MS consisting of 4.6 g/L MS salts and vitamins (Phyto Technology Laboratories, USA), 2% sucrose and 100 μM acetosyringone with an adjusted pH of 5.8. Injection of agrobacteria was performed as illustrated in the FIG. 2, multiple rounds of injections were applied to each sites. The injection sites were immediately covered with plastic saran wraps for 3 days. Plants were placed in the dark at 28° C. for two days, then removing the coverings and put the plants back to the high-light greenhouse.

Tissue culture media. Basic MS media consists of 4.6 g/L MS salts and vitamins, 30 g/L sucrose and 8 g/L agar with an adjusted pH of 5.8. Calluses and shoots induction media: MS+3 mg/l 6-BA+100 mg/l Kanamycin+100 mg/l Timentin, roots inducing media: 1/2 MS+3 mg/l 6-BA+50 mg/l Kanamycian+100 mg/l Timentin.

Detection of GFP fluorescence. GFP fluorescence was detected with a fluorescent microscope (Leica, German). The imaging of fluorescent signals was conducted with an imaging system (Nikon D800 Digital Camera) attached to the microscope.

PCR analysis. Genomic DNA was extracted from leaf tissues according to the CTAB method (Del et al. 1989). PCR with primers for amplifying GFP were preformed to detect transgenic events. PCR were carried out with Q5 Hot Start High-Fidelity DNA Polymerase (New England Biology, USA) according to its manual instruction. Amplified DNA fragments were separated by electrophoresis on 2% agarose gels which were stained with SYBR green (Thermo Scientific, USA) and visualized with the Omega Lum™ Imaging system (Gel Company, USA). The primers used to amply GFP was listed in Table 7.

Anthocyanin Determination. Anthocyanin was extracted from the shoots of T1 snapdragon seedlings. The extraction and determination of anthocyanin were modified according to the method described by (Neff and Chory 1998). In brief, five seedling stems were collected from T1 transgenic and wildtype snapdragons, respectively, and then ground to powder in the liquid N2 after weighting. Ground samples were incubated in 300 μL extraction solution (297 μL of Methanol and 3 μL HCl) overnight, then 200 μL Milli-Q H₂O and 500 μL of chloroform were added to each sample prior to spinning down the extract. 400 μL of supernatant was used and mixed with 237.6 μL of Methanol and 2.4 μL HCl+160 μL Milli-Q H₂O to bring the volume up to 800 μL. The absorbance of each sample was read at 530 nm and 657 nm using the spectrophotometer for anthocyanin determination. The blank should be 432 μL Methanol 48 μL HCl and 320 μL Milli-Q H₂O for a total of 800 μL.

Sudan-7B Staining. The calluses regenerated from Bok Choy explants after about 2-week culture on the MS media containing 3 mg/L 6-BA were used for Sudan Red 7B (Thermo Scientific, USA) staining according to a previously described method (Kadokura et al. 2018). In brief, the callus and seedling samples were dehydrated through a series of isopropanol (20%, 40%, 60%) for 20 minutes per treatment, then incubated in 60% isopropanol containing 0.5% Sudan Red 7B for 1 h. All samples were subsequently rehydrated through a reverse process and washed three times with distilled water before imaging. For seed staining, seed samples were first incubated in 15% commercial bleach containing 6% NaClO, and rotated in a shaker with 200 RPM at the room temperature until the seed embryo was isolated.

RNA extraction and RT-PCR analysis. To determine transcript levels of anthocyanin biosynthetic genes in snapdragon T1 seedlings and downstream genes of PLT5 in the transformed calluses of Bok Choy, total RNA was isolated from 100 mg of samples using RNAzol® RT RN190 (Molecular Research Center, USA). The synthesis of cDNA was performed using QuantiTect Reverse Transcription Kit (QIAGEN, Germany). The qRT-PCR reaction was composed of cDNA, primers and Power SYBR® Green Master Mix (Thermo Fisher, USA), and detected on a CFX96 Real-time system (BIO-RAD, USA). The primers used for examining the transcripts of anthocyanin biosynthetic genes including AmDEL (Accession number, M84913.1), AmCHS (Accession number, X03710), AmCHI (Accession number, AB861648), AmF3H (Accession number, LC194907), AmDFR (Accession number, P14721), and AmUBQ (ubiquitin) (Accession number, X67957) were designed according to the previous report (Fujino et al. 2018); the primers used for examining the transcripts of PLT5 down-stream genes including PLT1 (Accession number, XM_009111838.2), PLT2 (Accession number, XM_009149529.2), CUC1 (Accession number, XM_009118219.3), CUC2 (Accession number, XM_033282328.1), STM (Accession number, NC_024803.2), YUC4 (Accession number, LR031573.1) and the reference gene tubulin (Tub) (Accession number, D78496) were designed according to the previous studies (Qi et al. 2010; Mathew and Prasad 2021; Shin et al. 2020), and listed in the Table 7.

### Example 1. PLT5-Mediated De-Novo Shoot Regeneration from Aerial Organs of Antirrhinum majus

To examine the effect of developmental regulators on de-novo shoot regeneration from aerial organs, five different constructs containing Arabidopsis PLT5, ESR1, WUS-BBM, WUS and WIND1 under the CaMV35S promoter were used to test if they promote shoot regeneration in Snapdragon (Antirrhinum majus) (FIG. 1 a and b). To facilitate monitoring the transgene expression, a green fluorescent protein gene (cGFP) and an anthocyanin production regulator DELIA (DEL) from Snapdragon were included in all five constructs (FIG. 1 a and b). All plasmid DNA were initially transformed into A. tumefaciens strain GV3101 or EHA105.

First evaluated was how these DRs affect de-novo shoot regeneration in Snapdragon (Antirrhinum majus); Snapdraon is one of top fresh-cut and potted ornamental plants, and has long been used as a model plant species for studying plant development due to its substantial genetic diversity and well-established transposon mutagenesis system (Schwarz-Sommer et al. 2003; Lian et al. 2020; Dyer et al. 2007). However the lack of efficient genetic transformation pipeline is the major limiting factor for the full application of snapdragon in genetic and molecular studies.

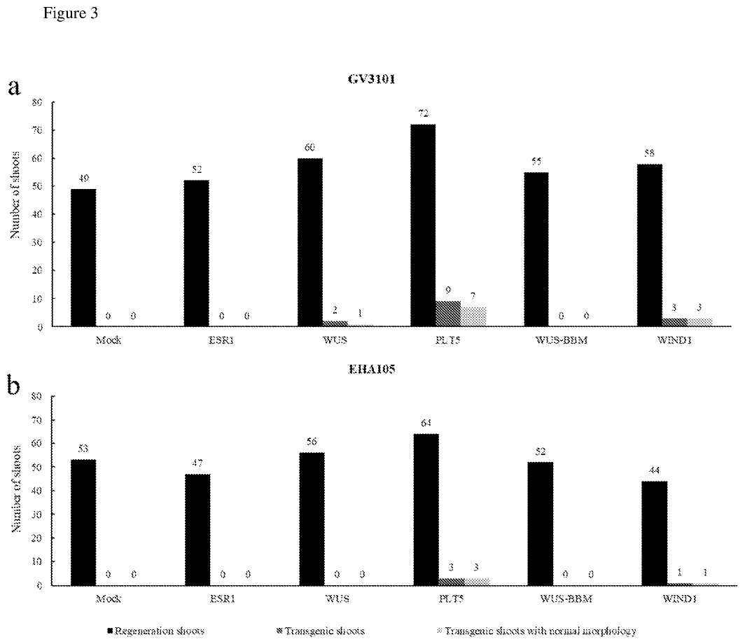

The primary and axially stems of approximate 70-days-old snapdragon plants when the flower bud differentiate initiated, were cut off (FIG. 2a), and agrobacteria solution containing different DR-constructs or a construct with no DR were injected at the indicated sites in the FIG. 2b. To our surprise, de-novo shoots were able to rapidly regenerate from the additional axillary shoot meristems near the wounding site when the control construct with no DR was injected (FIG. 2 d, and Table 1), but the frequency of regenerated shoots is much higher when GV3101 with different DRs were applied, with a highest shoot regeneration frequency (90%) and genetic transformation efficiency (12.5%) for PLT5 (Table 1). After genotyping with PCR for the presence of regenerated shoots, it was confirmed the WUS, PLT5 and WIND1 may facilitate genetic transformation, leading to transformation efficiency of 3.3%, 12.5%, and 5.17%, respectively when GV3101 strain was used. The infection with EHA105 caused a much lower transformation efficiency for both PLT5 and WIND1 and no transformation success for WUS (Table 1 and FIG. 3). And thus, GV3101 is an ideal agrobacterium strain for injection to improve snapdragon shoot regeneration and transformation efficiency, comparing with the EHA105.

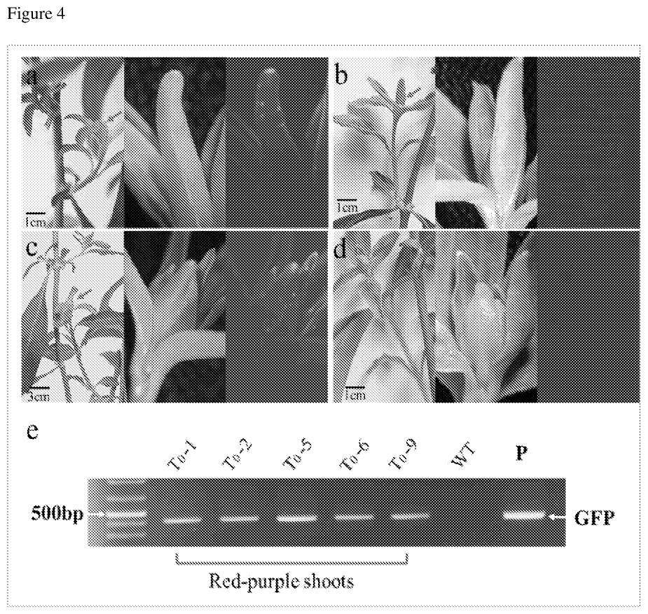

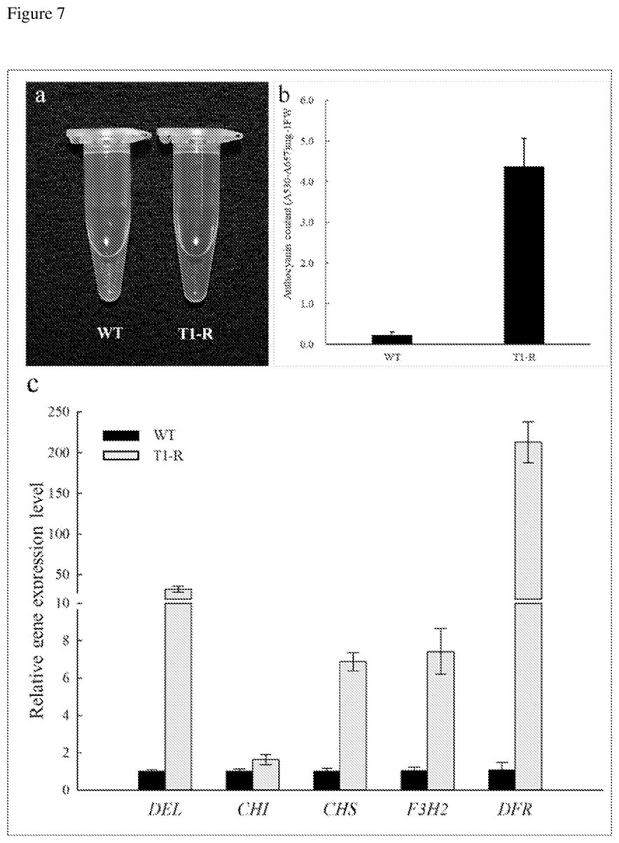

Given that the highest regeneration and transformation frequency caused by PLT5, more details were next given to its effect on de-novo shoot regeneration and genetic transformation. Unlike the control construct, the PLT5 promote development of embryonic-callus-like tissues at the wound sties 7 days after injection (FIG. 2c), and multiple shoots were subsequently regenerated from these callus-like tissues at 14 days after injection (FIG. 2d). Furthermore, the shoot stems exhibit red pigment due to the increased anthocyanin production (FIG. 2c, h, j. FIG. 7 a-b) at 25 days after injection; and more striking purple pigment appeared at adult stage of some shoots, while no red pigment was observed in the regenerated shoots after infection with control vectors (FIG. 2f-g. FIG. 7 a-b). Additionally, the DNA genotyping for GFP presence and detection of stable fluorescent signals further confirmed the successful integration of T-DNA into the plant genome in these transgenic shoots (FIG. 4a-e). One interesting observation was that almost all non-transgenic shoots emerge faster than the transgenic shoots that experienced the callus formation stage, indicating that the callus-formation was critical for successful genetic transformation.

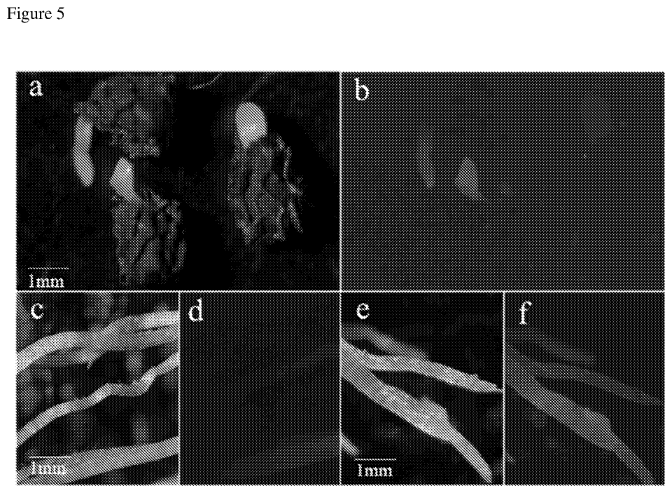

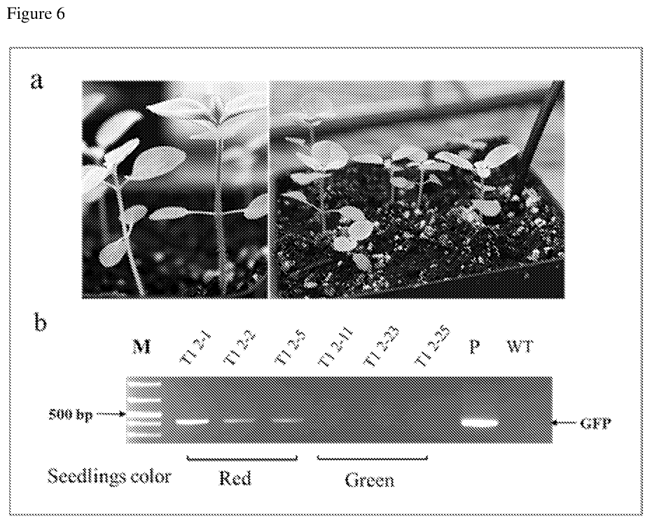

To examine if the transgenes can be stably inherited to next generation, GFP detection was performed in the T1 seedling, strong GFP fluorescent signals were detected in the radicles and roots in some segregated seeds (FIG. 5). In addition, the trait of red stem due to the transgene was also stably transmitted to T1 progenies (FIG. 6a). The PCR results confirmed that GFP was present in red-stem T1 seedlings but not the green ones (FIG. 6b). In consistence with this observation, transcript levels of DEL and other down-stream biosynthetic genes including chalcone isomerase (CHI), chalcone synthase (CHS), flavanone 3-hydroxylase (F3H2), dihydroflavonol 4-reductase (DFR) (Xie et al. 2016; Naing and Kim 2018) were 32, 1.6, 6.8, 7.4 and 212 folds higher than those in the control plants (FIG. 7c), Collectively, these results suggested that PLT5 promotes de-novo shoot regeneration at wound sites as well as stable genetic transformation.

### Example 2. PLT5 Promotes De-Novo Shoot Regeneration from Aerial Organs of Tomatoes

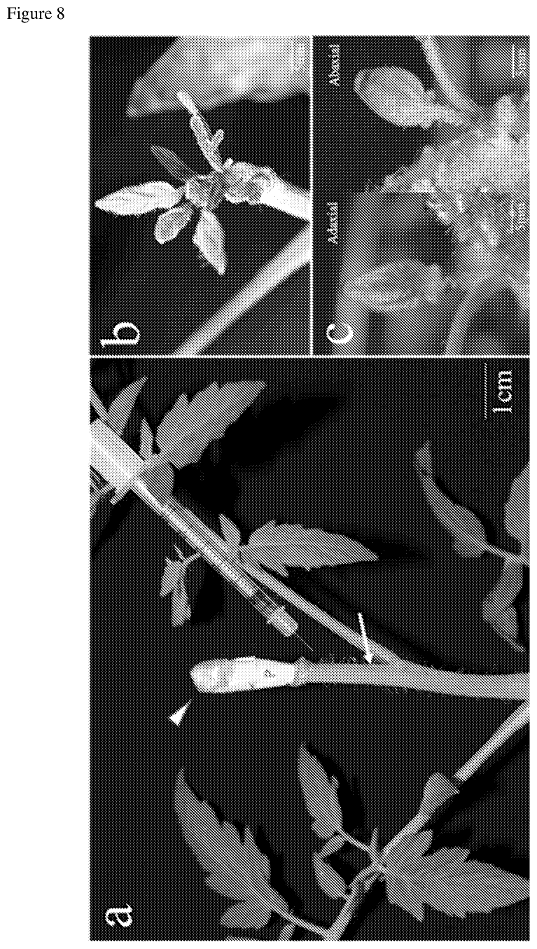

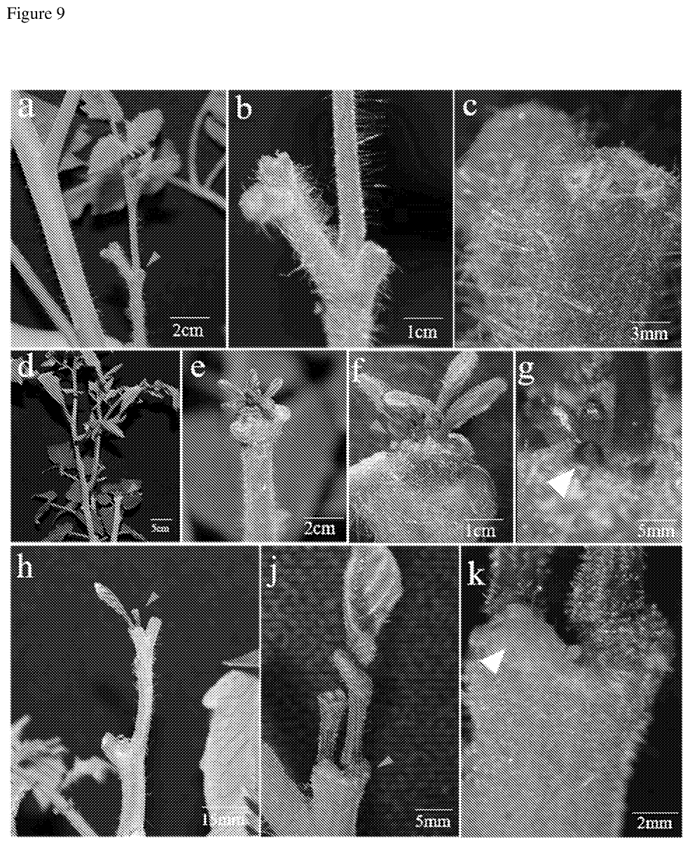

Based on the positive results from Snapdragon, the application of this method was extended to tomato to examine if WUS, WIND1 and PLT5 also promote on de-novo shoot regeneration and genetic transformation from wound sites in tomato. GV3101 containing the plasmid with PLT5, WUS1 and WIND1 were injected to the wound sites of stems as well as axillary meristem sites (FIG. 8a). Single green shoot typically regenerated from axillary meristems within 12 days after injection (FIG. 8 c, FIGS. 9a and b), and no visible callus tissue were developed at the wound sites (FIG. 9c). By contrast, embryonic-callus tissues were formed at the wound sites (FIG. 9 g and k), and the multiple red-purple shoots were able to regenerate from these callus after 20˜25 days injection (FIG. 9d-g).

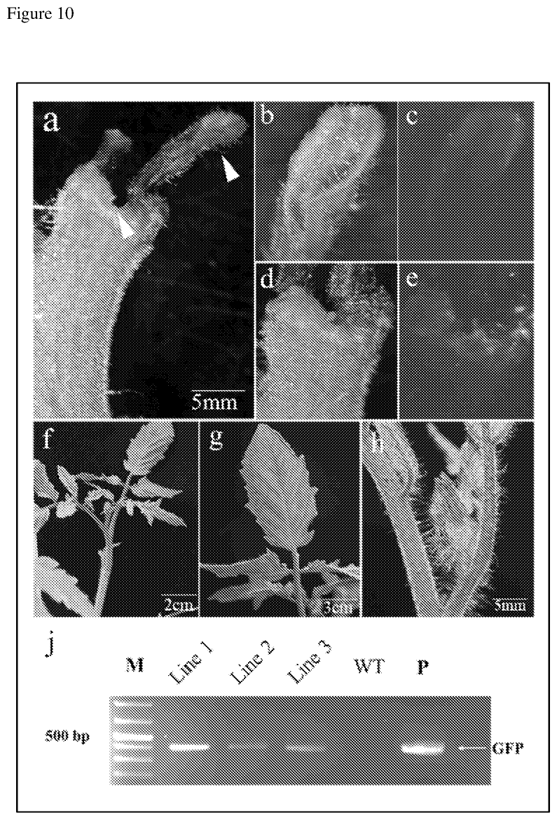

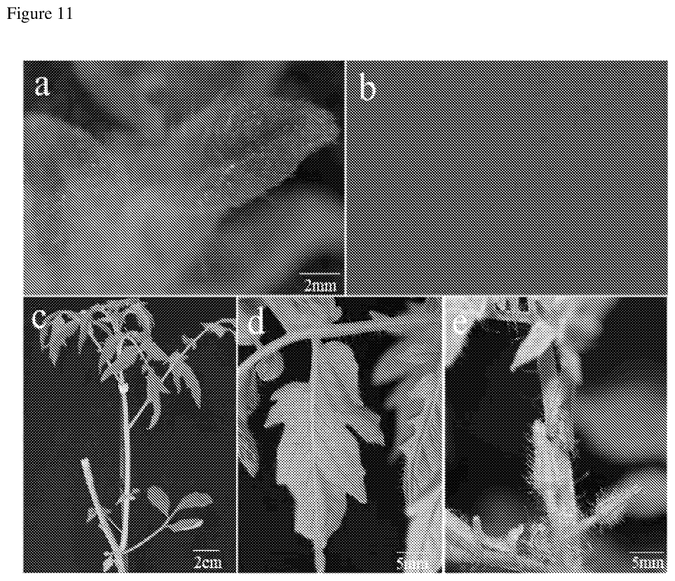

Microscopic examination showed that GFP fluorescence was not able to be detected in the shoots regenerated from axillary meristems (FIGS. 11a and b), while both callus-like tissue and newly regenerated shoot from the wound site display GFP fluorescence (FIG. 10a-e), indicating that these de-novo regenerated shoots from the wound sites are transgenic; PCR genotyping results also confirmed the presence of the transgene in these shoots (FIG. 10j). The transformation success was further corroborated by the notable anthocyanin accumulation in the mature shoots, abaxial side of leaves and flower sepals (FIG. 10f-h), while there was a lack of such pigment in non-transgenic shoots (FIG. 11c-e).

Surprisingly, all the transgenic tomato shoots were initiated from the wound sites, none of regenerated shoots from axillary meristems are transformed (Table 2). The developmental regulator PLT5 exhibited the best effect on improving shoot regeneration and transformation efficiency. After injection of plasmid with PLT5, three out of five regenerated shoots from wound sites were transgenic, whereas one transgenic shoot was obtained from two regenerated shoots after injected with plasmid containing WUS1, and no shoot was regenerated from the wound sites after injection of plasmid DNA with Wind1 or no DR (Table 2).

### Example 3. PLT5 Promotes De-Novo Shoot Regeneration and Genetic Transformation in Bok Choy and Sweet Pepper

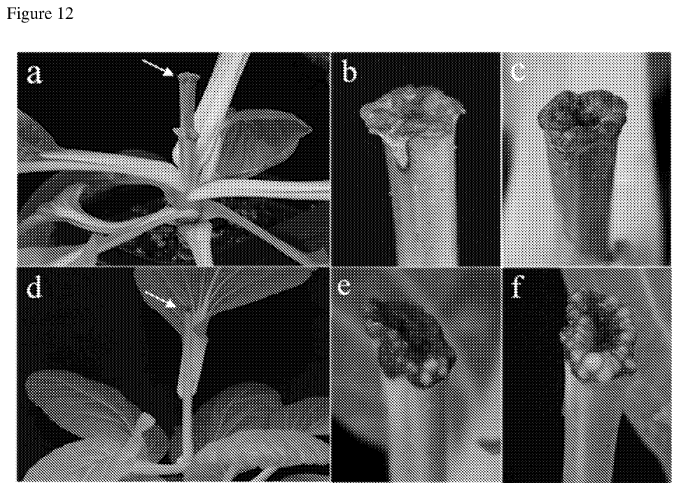

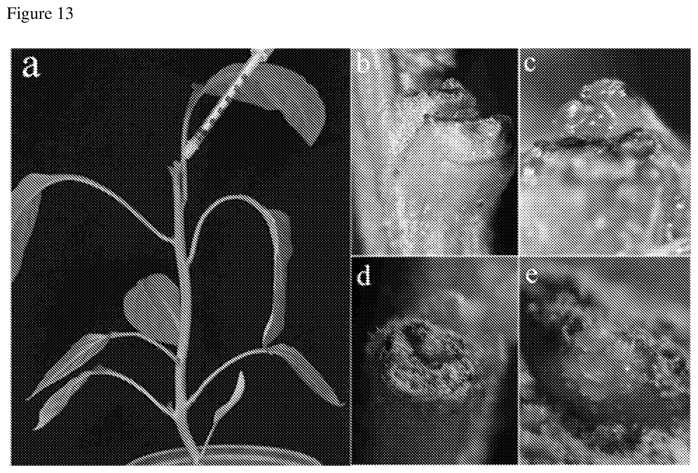

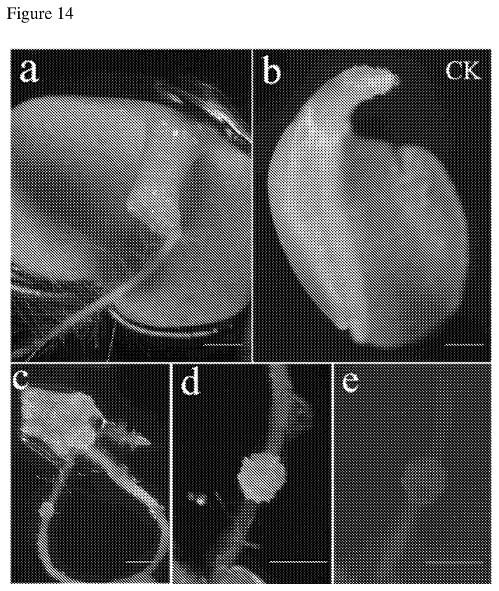

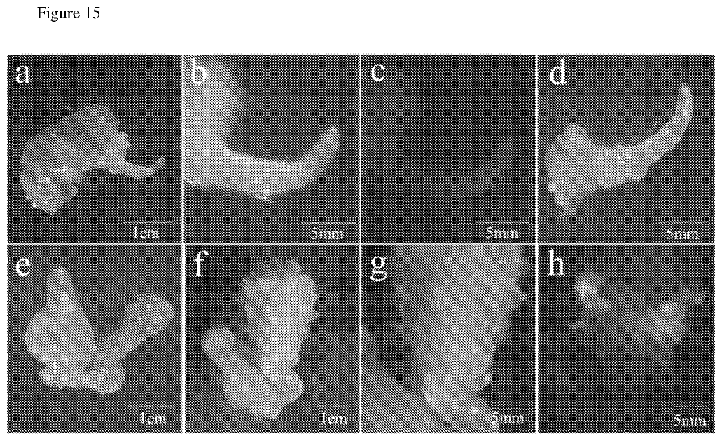

In addition to snapdragon and tomato, the method was tested in sweet pepper and Bok choy (Brassica rapa ssp chinensis), which are extremely recalcitrant to transformation. As showed in the FIG. 12 and FIG. 13, injection of GV3101 with PLT5 containing plasmid was able to promote callus or embryo-like structure formation, but no shoot could regenerate from these callus, possibly due to the quick deposition of suberin (FIGS. 12 and 13). Therefore, it was tested whether PLT5 promoted plant regeneration and genetic transformation through tissue culture. In the beginning, the cotyledons with petiole of Bok Choy were cultured on hormone-free MS medium. When the plasmid with PLT5 was applied for transformation, roots were able to initiate from the explants of Bok Choy within two weeks culture (FIG. 14a), but not happened in the explant infected with DR-free plasmid (FIG. 14b). With one more week of culture on the same media, the callus could initiate from these regenerated roots (FIG. 14 c and d), and the GFP could be detected in the regenerated root as well as the callus (FIG. 14 e). The new regenerated root tip with GFP was cut off from the callus, and placed on the new MS medium with 3 mg/L 6-BA addition. After 3 weeks of culture on the new media, the transformed embryo-like structure with GFP could be regenerated, and these embryos continued to develop into multiple shoots on the same medium containing 3 mg/L 6-BA (FIG. 15). Since the 6-BA had significantly improved the regeneration efficiency, we also attempted to place the explant (cotyledon with petiole) directly on the MS medium with 3 mg/L 6-BA. Surprisingly, although the root regeneration were significantly inhibited, the transformed shoots could be directly induced from the callus within one month (FIG. 16 b-d), but no shoot could regenerate from the callus without PLT5 infection (FIG. 16a).

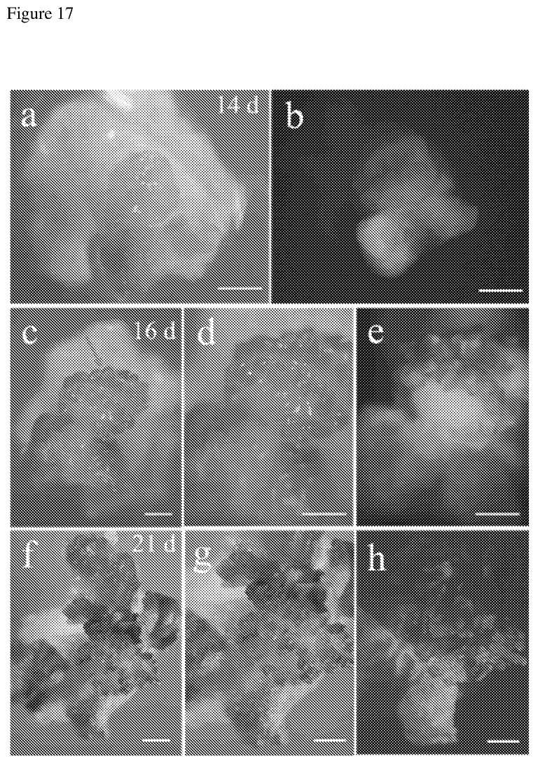

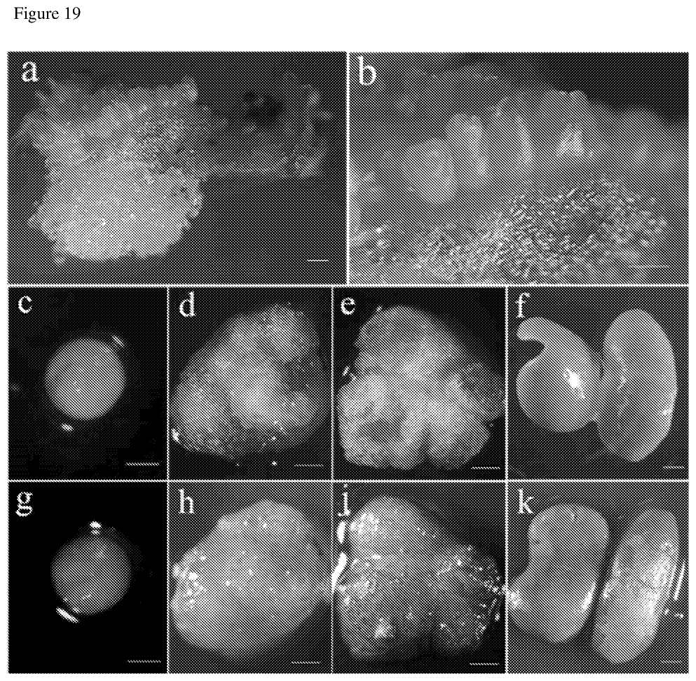

Interestingly, in some cases, it was found that only the part zone of regenerated callus turned green after 2 weeks culture on MS medium with 3 mg/L 6-BA when infected by agrobacterium containing PLT5 plasmid, and GFP signal was only detected on this green zone of callus instead of the whole callus (FIG. 16b and FIG. 17b). Subsequent shoots regeneration could only initiate from the green area as well (FIG. 16c-d and FIG. 17c-h), which strongly suggested PLT5 could promote the cellular differentiation, resulting in shoot meristem formation. In addition, the green zone of calluses with strong GFP signal and white calluses from control group with weak GFP signal at the same stage were collected as samples to detect the transcript level of PLT5 down-stream genes (FIGS. 18a and b). The qPCR result showed that PLT1, PLT2, CUC1, CUC2, STM and the auxin biosynthetic gene YUC4 were 7.6, 4.1, 3.3, 26.8, 15.5 and 1.8 folds higher in the calluses transformed with the PLT5 plasmid than those in the calluses infected with the control plasmid (FIG. 18c). With the closer observation, it was found that the multiple shoots initiated from the callus by the embryogenesis pathway (FIG. 17c and FIG. 19 a and b). To support this hypothesis, the calluses were stained with Sudan Red 7B, the result showed that the transformed callus had a darker red color, which was much closer with the color of seed embryo after staining comparing with the callus from the control group (FIG. 19 c-k).

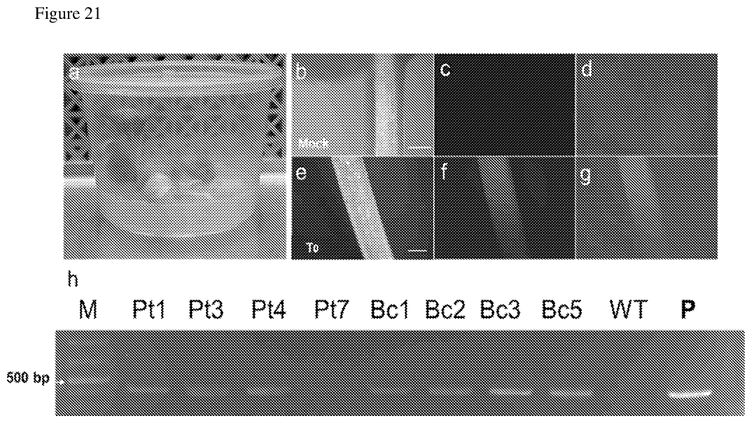

The methods herein were tested with an additional genotype of B. rapa cv Pei-Tsai. Similarly, plant regeneration and genetic transformation have been greatly improved by PLT5. Transgenic shoots with strong GFP signal were able to regenerate quickly from the cotyledon explants after infected with Agrobacteria containing PLT5 plasmid (FIG. 20 d-h), whereby only callus were formed when explants infected with Agrobacteria containing DR-free plasmid (FIG. 20 a and b). GFP fluorescence detection has confirmed the presence of transgenes in the transgenic plants but not the wild type plants (no plant was able to regenerate without aid of PLT5). Transformed seedlings in both B. rapa cabbages were further confirmed by the PCR genotyping (FIG. 21). The transformation efficiency by using the modified protocol were shown in Tables 3-5.

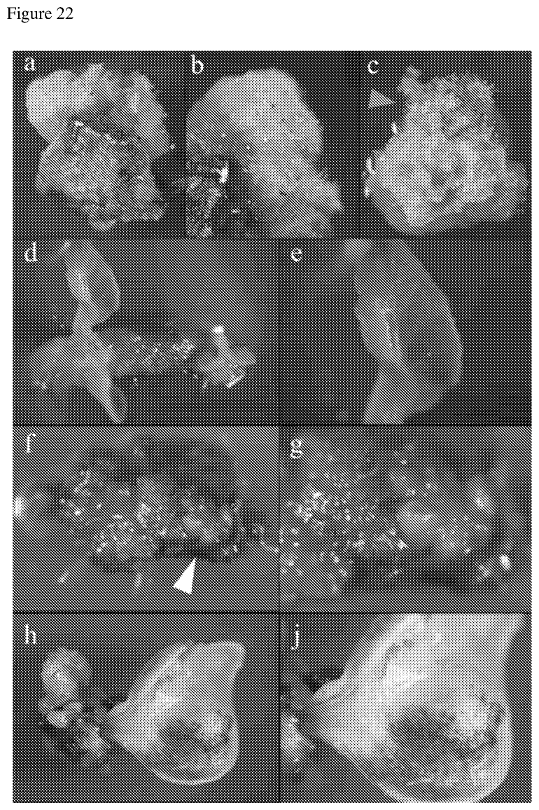

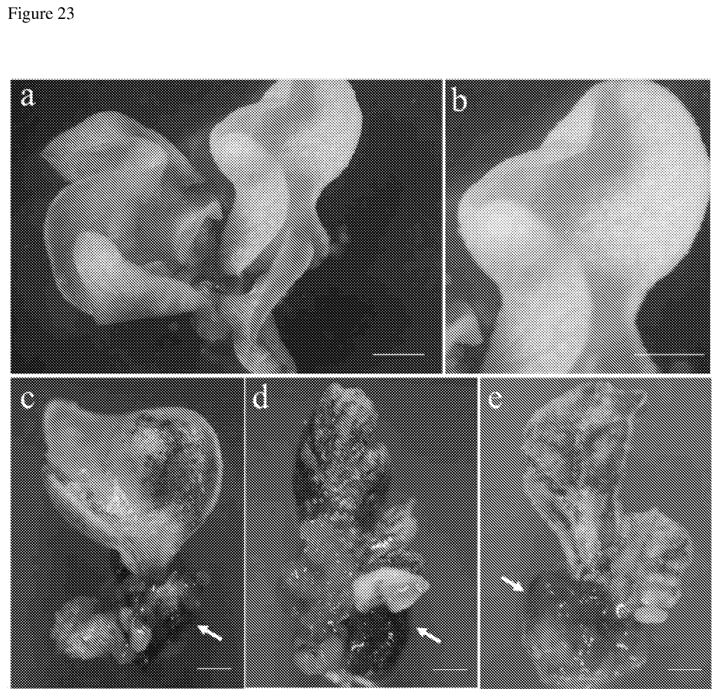

Lastly, PLT5 was tested for whether it promotes plant regeneration and genetic transformation in sweet pepper. Cotyledon explants (FIG. 22a) or hypocotyls (FIG. 22d) were infected with Agrobacterium containing PLT5 plasmid or DR-free plasmid. The medium was the same with the one used for B. rapa (i.e. MS medium with 3 mg/L 6-BA). Based on observations, no callus was formed and the shoots were directly regenerated from the hypocotyls, regardless of application of PLT5 plasmid or DR-free plasmid (FIG. 22d). However, no pigment could be detected in the regenerated shoots (FIG. 22 d and e). These shoots might initiate from the pre-exist meristem, and hypocotyls were not suitable for sweet pepper transformation. When cotyledons were used as explants, no shoot regenerated from calluses infected with the DR-free plasmid, although the pigment accumulation could be observed in the callus (FIG. 22a-c). By contrast, somatic embryos could regenerate from the regenerated calluses after 45 days infections with PLT5 plasmid (FIG. 22f-g), and shoots with various degrees of pigment slowly regenerated after further culturing these somatic embryos (FIG. 22 h-j and FIG. 23c-e). The transformation efficiency is shown in the Table 5.

TABLE 1
Effects of different DRs on shoot regeneration and genetic
transformation in soil-grown Snapdragon plants.

| | |
|---|---|
| GV3101 | EHA105 |

| | Regen- | Transfor- | Regen- | Transfor- |
|---|---|---|---|---|
| | eration | mation | eration | mation |
| | efficiency | efficiency | frequency | efficiency |
| DRs | (%) | (%) | (%) | (%) |
| Mock | 61.25 | 0.00 | 66.25 | 0.00 |
| ESR1 | 65.00 | 0.00 | 58.75 | 0.00 |
| WUS | 75.00 | 3.30 | 70.00 | 0.00 |
| PLT5 | 90.00 | 12.50 | 80.00 | 4.69 |
| WUS- | 68.75 | 0.00 | 65.00 | 0.00 |
| BBM | | | | |
| WIND1 | 72.50 | 5.17 | 55.00 | 2.27 |

Regeneration frequency: the number of regenerated shoots/Injection sites; Transformation efficiency: the number of transgenic shoots/the total regenerated shoots.

TABLE 2
Effects of different DRs on shoot regeneration and transformation
efficiency in soil-grown tomato plants

| | | Regeneration | | | |
|---|---|---|---|---|---|
| | | shoots From | Trans- | Regeneration | Trans- |
| | Injection | axillary | genic | shoots from | genic |
| DRs | sites | meristems | shoots | wound sties | shoots |
| Mock | 25 | 13 | 0 | 0 | 0 |
| WUS | 25 | 11 | 0 | 2 | 1 |
| PLT5 | 25 | 7 | 0 | 5 | 3 |
| WIND1 | 25 | 9 | 0 | 0 | 0 |

TABLE 3
Effects of PLT5 on regeneration and transformation in Bok Choy
when using the regenerated transformed roots to induce shoots.

| | | | The number of | |
|---|---|---|---|---|
| | The number | The number | embryonic callus | Transgenic |
| Treatment | of explants | of roots | with GFP | shoots |
| Control | 20*3 | 0.0 ± 0.0 | 0.0 ± 0.0 | 0.0 ± 0.0 |
| PLT5 | 20*3 | 2.7 ± 0.6 | 1.3 ± 0.6 | 3.7 ± 1.5 |

Twenty explants were first cultured on phytohormone-free MS media to induce root, then the root tip was cut off and placed on shoot-induction MS medium with 3 mg/L 6-BA addition.
The experiment was repeated three times.
Values = Mean ± SD.

TABLE 4
Effects of PLT5 on shoot regeneration and genetic
transformation in Bok Choy and Pei-Tsai bypass
the roots regeneration in the beginning.

| | | The number | The number of | Transgenic |
|---|---|---|---|---|
| Varieties | Treatment | of explants | embryonic callus | shoots |
| Bok Choy | Control | 25*4 | 0.0 ± 0.0 | 0.0 ± 0.0 |
| | PLT5 | 25*4 | 2.0 ± 0.8 | 6.3 ± 3.0 |
| Pei-Tsai | Control | 25*4 | 0.0 ± 0.0 | 0.0 ± 0.0 |
| | PLT5 | 25*4 | 1.5 ± 1.3 | 4.0 ± 3.4 |

Twenty five explants were cultured on MS medium with 3 mg/L 6-BA addition.
The experiment was repeated four times.
Values = Mean ± SD.

TABLE 5
Effects of PLT5 on the regeneration
and transformation in sweet pepper

| | The number | The number of | Transgenic |
|---|---|---|---|
| Treatment | of explants | embryonic callus | shoots |
| Control | 80 | 0 | 0 |
| PLT5 | 80 | 3 | 4 |

TABLE 6
The primers used for building
POX135-DRs-DEL vector

| | | SEQ |
|---|---|---|
| | | ID |
| Primer names | Primers (5′-3′) | NO: |
| NOSterSpeI-F | atactagtGAATTTCCCCGATCGTTC | 1 |
| NOSterHindIII-R | ataagcttAGTTAGCTCACTCATTAGGC | 2 |
| AtESRAscIF3 | atggcgcgccATGGAAAAAGCCTTGAGAAAC | 3 |
| AtESRBamHR3 | atggatcCTATCCCCACGATCTTCG | 4 |
| AtWIND1AscIF3 | atggcgcgccATGGCAGCTGCTATGAATT | 5 |
| AtWIND1BamHR3 | atggatcCTAAGCTAGAATCGAATCCC | 6 |
| 35S-AatII-F | agacgtCTCTGCCGACAGTGGTC | 7 |
| NOSterMluI-R | taacgcgTAGTTAGCTCACTCATTAGGCACC | 8 |

TABLE 7
The primers used for qRT-PCR analysis and transformation test (GFP)

| Gene | | |
|---|---|---|
| name | Primers (5′-3′) | Purpose |
| AmUBQ | Fw-CGTGAAGACCTTGACTGGGAAGACG (SEQ ID NO: 9) | QRT-PCR analysis for |
| | Rv-CAAGGTGAAGAGTCGACTCCTTCTGG (SEQ ID NO: 10) | the relative expression |
| AmDEL | Fw-CAAGGGTTGCCTGGAAGAAC (SEQ ID NO: 11) | level of Del down-stream |
| | Rv-TTGCTCCCAGCTCAACTACA (SEQ ID NO: 12) | genes in the transformed |
| AmCHS | Fw-GAGGTTCCTCGTCTCGG (SEQ ID NO: 13) | snapdragon T1 seedlings. |
| | Rv-CACCGTACCACCAGCAAAG (SEQ ID NO: 14) | |
| AmCHI | Fw-GATCCAGGGGAAGTTCATC (SEQ ID NO: 15) | |
| | Rv-CGTCAATGGCAGTATCATG (SEQ ID NO: 16) | |
| AmF3H | Fw-GCTTCGTTTCGACATGTCG (SEQ ID NO: 17) | |
| | RV-AGTTTCATCAACTCCTCGCTATAC (SEQ ID NO:1 8) | |
| AmDFR | Fw-CTGTTTCCAATTTCGGAGG (SEQ ID NO: 19) | |
| | Rv-TTTGCAATGTCTGGAAGCAG (SEQ ID NO: 20) | |
| PLT1 | Fw-ACTTTGTCCATGGGGACCAC (SEQ ID NO: 21) | QRT-PCR analysis for the |
| | Rv-CCGTCTCAACAACGGCTAGT (SEQ ID NO: 22) | relative expression level |
| PLT2 | Fw-AGCAACCCTGGAATGCTTCA (SEQ ID NO: 23) | of PLT5 down-stream genes |
| | Rv-CCGTGGAAAACTCATCCCCA (SEQ ID NO: 24) | in the transformed |
| CUC1 | Fw-TGCATGAGTATCGCCTCGAC (SEQ ID NO: 25) | Bok choy callus. |
| | RV-TCGTCTGCTCTCTACCGACA (SEQ ID NO: 26) | Genotyping for transgenic |
| CUC2 | Fw-CGCAAGGTTCTTGACGGTTG (SEQ ID NO: 27) | conformation. |
| | RV-GTCGGATACTTCCGGTCACG (SEQ ID NO: 28) | |
| STM | Fw-CCTCCGTCTCAAGAAGCGAA (SEQ ID NO: 29) | |
| | RV-GGATTGAGTCCTCCTGCGAG (SEQ ID NO: 30) | |
| YUC4 | Fw-TACACAGCGTCTTGCGTCTT (SEQ ID NO: 31) | |
| | RV-GAGTCTTACGCTGCCCGATT (SEQ ID NO: 32) | |
| TUB | Fw-ATGTTTAGGAGGGTGAGTGAGCA (SEQ ID NO: 33) | |
| | RV-CTCTCGGCTTCTGTAAACTCCAT (SEQ ID NO: 34) | |
| GFP | Fw-ACAAGTTCAGCGTGTCCG (SEQ ID NO: 35) | |
| | RV-TCACCTTGATGCCGTTCT (SEQ ID NO: 36) | |

### REFERENCES

Altpeter F. Springer N M, Bartley L E, Blechl A E, Brutnell T P, Citovsky V. Conrad L J, Gelvin S B. Jackson D P, Kausch A P, Lemaux P G, Medford J I, Orozco-Cardenas M L, Tricoli D M, Van Eck J, Voytas D F, Walbot V, Wang K, Zhang Z J, Stewart C N, Jr. (2016) Advancing Crop Transformation in the Era of Genome Editing. Plant Cell 28 (7): 1510-1520. doi: 10.1105/tpc.16.00196
Ariga H. Toki S, Ishibashi K (2020) Potato Virus X Vector-Mediated DNA-Free Genome Editing in Plants. Plant and Cell Physiology 61 (11): 1946-1953. doi: 10.1093/pcp/pcaa123
Boutilier K, Offringa R, Sharma V K, Kieft H, Ouellet T, Zhang L M, Hattori J, Liu C M, van Lammeren A A M, Miki B L A, Custers J B M, Campagne M M V (2002) Ectopic expression of BABY BOOM triggers a conversion from vegetative to embryonic growth. Plant Cell 14 (8): 1737-1749. doi: 10.1105/tpc.001941
Brown D C. Thorpe T A (1986) Plant regeneration by organogenesis. Cell culture and somatic cell genetics of plants 3:49-65
Clough S J, Bent A F (1998) Floral dip: a simplified method for Agrobacterium-mediated transformation of Arabidopsis thaliana. Plant J 16 (6): 735-743. doi: 10.1046/j.1365-313x.1998.00343.x
Collado R, Bermúdez-Caraballoso I, García L, Veitía N, Torres D, Romero C, Angenon G (2016) Epicotyl sections as targets for plant regeneration and transient transformation of common bean using Agrobacterium tumefaciens. In Vitro Cellular & Developmental Biology-Plant 52 (5): 500-511
de Melo B P, Lourenco-Tessutti I T, Morgante C V, Santos N C, Pinheiro L B, de Jesus Lins C B, Silva M C M, Macedo L L P. Fontes E P B, Grossi-de-Sa M F (2020) Soybean embryonic axis transformation: combining biolistic and Agrobacterium-mediated protocols to overcome typical complications of in vitro plant regeneration. Frontiers in Plant Science 11
Debernardi J M, Tricoli D M, Ercoli M F, Hayta S, Ronald P. Palatnik J F, Dubcovsky J (2020) A GRF-GIF chimeric protein improves the regeneration efficiency of transgenic plants. Nat Biotechnol 38 (11): 1274-1279. doi: 10.1038/s41587-020-0703-0
Del G S. Manfioletti G, Schneider C (1989) The CTAB-DNA precipitation method: a common mini-scale preparation of template DNA from phagemids, phages or plasmids suitable for sequencing. Biotechniques 7 (5): 514-520
Dinesh-Kumar S P, Voytas D F (2020) Editing through infection. Nat Plants 6 (7): 738-739. doi: 10.1038/s41477-020-0716-1
Dyer A G, Whitney H M, Arnold S E, Glover B J, Chittka L (2007) Mutations perturbing petal cell shape and anthocyanin synthesis influence bumblebee perception of Antirrhinum majus flower colour. Arthropod-Plant Interactions 1 (1): 45-55
Ellison E E, Nagalakshmi U, Gamo M E, Huang P J, Dinesh-Kumar S, Voytas D F (2020) Multiplexed heritable gene editing using RNA viruses and mobile single guide RNAs. Nat Plants 6 (6): 620-+. doi: 10.1038/s41477-020-0670-y
Fatihah H N, López D M, van Arkel G, Schaart J G, Visser R G, Krens F A (2019) The ROSEA1 and DELILA transcription factors control anthocyanin biosynthesis in Nicotiana benthamiana and Lilium flowers. Scientia Horticulturae 243:327-337
Fujino N, Tenma N, Waki T, Ito K, Komatsuzaki Y, Sugiyama K, Yamazaki T, Yoshida S, Hatayama M. Yamashita S (2018) Physical interactions among flavonoid enzymes in snapdragon and torenia reveal the diversity in the flavonoid metabolon organization of different plant species. The Plant Journal 94 (2): 372-392
Gaillochet C, Lohmann J U (2015) The never-ending story: from pluripotency to plant developmental plasticity. Development 142 (13): 2237-2249
Heidmann I, De Lange B, Lambalk J, Angenent G C, Boutilier K (2011) Efficient sweet pepper transformation mediated by the BABY BOOM transcription factor. Plant cell reports 30 (6): 1107-1115
Hoerster G, Wang N, Ryan L, Wu E, Anand A, McBride K, Lowe K, Jones T, Gordon-Kamm B (2020) Use of non-integrating Zm-Wus2 vectors to enhance maize transformation. In Vitro Cellular & Developmental Biology-Plant 56 (3): 265-279
Ikeuchi M, Favero D S, Sakamoto Y, Iwase A, Coleman D, Rymen B, Sugimoto K (2019) Molecular mechanisms of plant regeneration. Annual review of plant biology 70:377-406
Ikeuchi M, Iwase A, Rymen B, Lambolez A, Kojima M, Takebayashi Y, Heyman J, Watanabe S, Seo M, De Veylder L (2017) Wounding triggers callus formation via dynamic hormonal and transcriptional changes. Plant physiology 175 (3): 1158-1174
Ikeuchi M, Ogawa Y, Iwase A, Sugimoto K (2016) Plant regeneration: cellular origins and molecular mechanisms. Development 143 (9): 1442-1451
Iwase A, Harashima H, Ikeuchi M, Rymen B, Ohnuma M, Komaki S, Morohashi K, Kurata T, Nakata M, Ohme-Takagi M (2017) WIND1 promotes shoot regeneration through transcriptional activation of ENHANCER OF SHOOT REGENERATION1 in Arabidopsis. The Plant Cell 29 (1): 54-69
Iwase A, Mita K. Nonaka S, Ikeuchi M, Koizuka C, Ohnuma M, Ezura H, Imamura J, Sugimoto K (2015) WIND1-based acquisition of regeneration competency in Arabidopsis and rapeseed. J Plant Res 128 (3): 389-397. doi: 10.1007/s10265-015-0714-y
Iwase A. Mitsuda N, Ikeuchi M, Ohnuma M, Koizuka C, Kawamoto K, Imamura J, Ezura H, Sugimoto K (2013) Arabidopsis WIND1 induces callus formation in rapeseed, tomato, and tobacco. Plant Signal Behav 8 (12): e27432. doi: 10.4161/psb.27432
Iwase A, Mitsuda N, Koyama T, Hiratsu K, Kojima M, Arai T, Inoue Y, Seki M, Sakakibara H, Sugimoto K (2011) The AP2/ERF transcription factor WIND1 controls cell dedifferentiation in Arabidopsis. Current Biology 21 (6): 508-514
Jones T, Lowe K, Hoerster G, Anand A, Wu E, Wang N, Arling M, Lenderts B, Gordon-Kamm W (2019) Maize transformation using the morphogenic genes Baby Boom and Wuschel2. In: Transgenic Plants. Springer, pp 81-93
Kadokura S, Sugimoto K, Tarr P. Suzuki T, Matsunaga S (2018) Characterization of somatic embryogenesis initiated from the Arabidopsis shoot apex. Developmental biology 442 (1): 13-27
Kareem A, Durgaprasad K. Sugimoto K, Du Y, Pulianmackal A J, Trivedi Z B, Abhayadev P V, Pinon V, Meyerowitz E M, Scheres B (2015) PLETHORA genes control regeneration by a two-step mechanism. Current Biology 25 (8): 1017-1030
Krizek B A (2015) AlNTEGUMENTA-LIKE genes have partly overlapping functions with AlNTEGUMENTA but make distinct contributions to Arabidopsis thaliana flower development. Journal of experimental botany 66 (15): 4537-4549
Lian Z, Nguyen C D, Wilson S, Chen J, Gong H, Huo H (2020) An efficient protocol for Agrobacterium-mediated genetic transformation of Antirrhinum majus. Plant Cell, Tissue and Organ Culture (PCTOC) 142 (3): 527-536
Lloyd A, Brockman A, Aguirre L, Campbell A, Bean A, Cantero A, Gonzalez A (2017) Advances in the MYB-bHLH-W D repeat (MBW) pigment regulatory model: Addition of a WRKY factor and co-option of an anthocyanin MYB for betalain regulation. Plant and Cell Physiology 58 (9): 1431-1441
Lotan T, Ohto M, Yee K M, West M A L, Lo R, Kwong R W, Yamagishi K, Fischer R L, Goldberg R B, Harada J J (1998) Arabidopsis LEAFY COTYLEDON1 is sufficient to induce embryo development in vegetative cells. Cell 93 (7): 1195-1205. doi: Doi 10.1016/S0092-8674 (00) 81463-4
Lowe K, Wu E, Wang N, Hoerster G, Hastings C, Lenderts B, Chamberlin M, Arling M, Anand A, Annalura V Use of the Maize Morphogenic Genes BBM and WUS2 to Improve Monocot Transformation. In: IN VITRO CELLULAR & DEVELOPMENTAL BIOLOGY-ANIMAL, 2016. SPRINGER 233 SPRING S T, NEW YORK, N Y 10013 USA, pp S5-S5
Ma X N, Zhang X Y, Liu H M, Li Z H (2020) Highly efficient DNA-free plant genome editing using virally delivered CRISPR-Cas9. Nat Plants 6 (7): 773-+. doi: 10.1038/s41477-020-0704-5
Maher M F, Nasti R A, Vollbrecht M, Starker C G, Clark M D, Voytas D F (2020) Plant gene editing through de novo induction of meristems. Nature biotechnology 38 (1): 84-89
Martins P K, Nakayama T J, Ribeiro A P, Cunha B, Nepomuceno A L, Harmon F G, Kobayashi A K, Molinari H B C (2015) Setaria viridis floral-dip: A simple and rapid Agrobacterium-mediated transformation method. Biotechnol Rep (Amst) 6:61-63. doi: 10.1016/j.btre.2015.02.006
Mathew M M, Prasad K (2021) Model systems for regeneration: Arabidopsis. Development 148 (6): dev 195347
Melnyk C W, Schuster C, Leyser O, Meyerowitz E M (2015) A developmental framework for graft formation and vascular reconnection in Arabidopsis thaliana. Current Biology 25 (10): 1306-1318
Mukeshimana G, Ma Y, Walworth A E, Song G-q, Kelly J D (2013) Factors influencing regeneration and Agrobacterium tumefaciens-mediated transformation of common bean (Phaseolus vulgaris L.). Plant biotechnology reports 7 (1): 59-70
Naing A H, Kim C K (2018) Roles of R2R3-MYB transcription factors in transcriptional regulation of anthocyanin biosynthesis in horticultural plants. Plant molecular biology 98 (1): 1-18
Naing A H, Park K I, Ai T N, Chung M Y, Han J S, Kang Y-W, Lim K B, Kim C K (2017) Overexpression of snapdragon Delila (Del) gene in tobacco enhances anthocyanin accumulation and abiotic stress tolerance. Bmc Plant Biol 17 (1): 65
Nakagawa T, Suzuki T, Murata S, Nakamura S, Hino T, Maeo K, Tabata R, Kawai T, Tanaka K, Niwa Y (2007) Improved Gateway binary vectors: high-performance vectors for creation of fusion constructs in transgenic analysis of plants. Bioscience, biotechnology, and biochemistry 71 (8): 2095-2100
Nasti R A, Voytas D F (2021) Attaining the promise of plant gene editing at scale. Proceedings of the National Academy of Sciences 118 (22)
Neff M M, Chory J (1998) Genetic interactions between phytochrome A. phytochrome B, and cryptochrome 1 during Arabidopsis development. Plant physiology 118 (1): 27-35
Nelson-Vasilchik K, Hague J, Mookkan M, Zhang Z J, Kausch A (2018) Transformation of Recalcitrant Sorghum Varieties Facilitated by Baby Boom and Wuschel2. Curr Protoc Plant Biol 3 (4): e20076. doi: 10.1002/cppb.20076
Nguyen C D, Li J, Mou B, Gong H. Huo H (2021) A case study of using an efficient CRISPR/Cas9 system to develop variegated lettuce. Vegetable Research 1 (1): 1-10
Prasad K, Grigg S P, Barkoulas M, Yadav R K, Sanchez-Perez G F, Pinon V, Blilou I, Hofhuis H, Dhonukshe P. Galinha C (2011) Arabidopsis PLETHORA transcription factors control phyllotaxis. Current Biology 21 (13): 1123-1128
Radhakrishnan D, Shanmukhan A P. Kareem A, Aiyaz M, Varapparambathu V. Toms A, Kerstens M, Valsakumar D, Landge A N, Shaji A (2020) A coherent feed-forward loop drives vascular regeneration in damaged aerial organs of plants growing in a normal developmental context. Development 147 (6): dev185710
Saha P. Blumwald E (2016) Spike-dip transformation of Setaria viridis. Plant J 86 (1): 89-101. doi: 10.1111/tpj.13148
Schwarz-Sommer Z. Davies B, Hudson A (2003) An everlasting pioneer: the story of Antirrhinum research. Nature Reviews Genetics 4 (8): 655-664
Schwinn K, Venail J, Shang Y, Mackay S, Alm V, Butelli E, Oyama R, Bailey P. Davies K. Martin C (2006) A small family of MYB-regulatory genes controls floral pigmentation intensity and patterning in the genus Antirrhinum. The Plant Cell 18 (4): 831-851
Shin J, Bae S. Seo P J (2020) De novo shoot organogenesis during plant regeneration. Journal of experimental botany 71 (1): 63-72
Sun H, Zhou N, Wang H, Huang D, Lang Z (2017) Processing and targeting of proteins derived from polyprotein with 2A and LP4/2A as peptide linkers in a maize expression system. PLOS One 12 (3): e0174804
Tsuwamoto R, Yokoi S, Takahata Y (2010) Arabidopsis EMBRYOMAKER encoding an AP2 domain transcription factor plays a key role in developmental change from vegetative to embryonic phase. Plant molecular biology 73 (4-5): 481-492
Wang C, Cui H-M, Huang T-H, Liu T-K, Hou X-L, Li Y (2016) Identification and validation of reference genes for R T-qPCR analysis in non-heading Chinese cabbage flowers. Frontiers in plant science 7:811
Wang M, Gao S L, Zeng W Z, Yang Y Q, Ma J F, Wang Y (2020) Plant Virology Delivers Diverse Toolsets for Biotechnology. Viruses-Basel 12 (11). doi: ARTN 1338 10.3390/v12111338
Xie Y, Tan H, Ma Z, Huang J (2016) DELLA proteins promote anthocyanin biosynthesis via sequestering MYBL2 and JAZ suppressors of the MYB/bHLH/WD40 complex in Arabidopsis thaliana. Molecular Plant 9 (5): 711-721
Yano R, Kanno Y, Jikumaru Y, Nakabayashi K, Kamiya Y, Nambara E (2009) CHOTTO1, a putative double APETALA2 repeat transcription factor, is involved in abscisic acid-mediated repression of gibberellin biosynthesis during seed germination in Arabidopsis. Plant physiology 151 (2): 641-654
Ye B-B, Shang G-D, Pan Y, Xu Z-G, Zhou C-M, Mao Y-B, Bao N, Sun L, Xu T, Wang J-W (2020) AP2/ERF transcription factors integrate age and wound signals for root regeneration. The Plant Cell 32 (1): 226-241
Zhang X, Xu G, Cheng C, Lei L, Sun J, Xu Y, Deng C, Dai Z, Yang Z, Chen X (2021) Establishment of an Agrobacterium-mediated genetic transformation and CRISPR/Cas9-mediated targeted mutagenesis in Hemp (Cannabis Sativa L.). Plant biotechnology journal
Zhao X Y, Su Y H, Cheng Z J, Zhang X S (2008) Cell fate switch during in vitro plant organogenesis. Journal of Integrative Plant Biology 50 (7): 816-824
Zou X, Li D, Luo X, Luo K, Pei Y (2008) An improved procedure for Agrobacterium-mediated transformation of trifoliate orange (Poncirus trifoliata L. Raf.) via indirect organogenesis. In Vitro Cellular & Developmental Biology-Plant 44 (3): 169-177
Zuo J R. Niu Q W. Frugis G. Chua N H (2002) The WUSCHEL gene promotes vegetative-to-embryonic transition in Arabidopsis. Plant J 30 (3): 349-359. doi: DOI 10.1046/j.1365-313X.2002.01289.x

What is claimed is:

1. A vector comprising, as operably linked components, an optional first expression cassette which comprises a nucleic acid sequence encoding at least one selectable marker, an optional second expression cassette which comprises a cloning site, a third expression cassette which comprises at least one DR DNA sequence that encodes PLT, WUS1, WIND1 and/or BBM-WUS, and an optional fourth expression cassette comprising a gene of interest.

2. The vector of claim 1, wherein the selectable marker encodes an antibiotic resistance selectable marker and/or an antibiotic-free selectable marker.

3. The vector of claim 2, wherein the antibiotic resistance selectable marker is NPTII.

4. The vector of claim 2, wherein the antibiotic-free selectable marker is GFP.

5. The vector of claim 1, wherein the cloning site is a multiple cloning site.

6. The vector of claim 1, wherein the first expression cassette comprises a CsVMV promoter region.

7. The vector of claim 1, wherein the second expression cassette comprises at least two CaMV35S promoter regions.

8. The vector of claim 1, wherein the third expression cassette comprises a CaMV35S promoter region.

9. The vector of claim 1, wherein PLT and WIND1 are cloned from plants.

10. The vector of claim 1, wherein the vector is competent for stable transformation of a plant genome.

11. A method of transforming a plant to express at least one heterologous nucleic acid sequence comprising delivering to the plant at least one agrobacterium comprising the vector of any of claim 1-10 or 27, the vector engineered to comprise at least one transgene, wherein the delivering results in introduction of the at least one transgene into at least one plant cell of the plant such that the at least one transgene is stably integrated into a genome of said at least one plant cell, or optionally, wherein delivering to the plant at least one agrobacterium comprising the vector of any of claim 1-10 or 27 comprises delivering two or more agrobacteria, wherein an agrobacterium of the two or more agrobacteria comprises a different vector comprising a different DR DNA sequence relative to another agrobacterium of the two or more agrobacteria.

12. The method of claim 11, wherein the plant is an adult plant.

13. The method of claim 11, wherein the plant is a recalcitrant plant

14. The method of claim 11, wherein the agrobacterium is suspended in an induction media.

15. The method of claim 14, wherein the induction media is selected from the group consisting of callus induction media, shoot induction media, or root induction media.

16. A method of promoting de novo shoot regeneration in a plant, comprising delivering to the plant at least one agrobacterium comprising the vector of any of claim 1-10 or 27, the vector engineered to comprise at least one transgene, wherein the delivering results in introduction of the at least one transgene into at least one plant cell of the plant, wherein the expression of the at least one DR DNA sequence increases de novo shoot regeneration in the plant compared to natural plant cells lacking the at least one DR DNA sequence, or optionally, wherein delivering to the plant at least one agrobacterium comprising the vector of any of claim 1-10 or 27 comprises delivering two or more agrobacteria, wherein an agrobacterium of the two or more agrobacteria comprises a different vector comprising a different DR DNA sequence relative to another agrobacterium of the two or more agrobacteria.

17. The method of claim 16, wherein the plant is an adult plant.

18. The method of claim 16, wherein the vector of any of claim 1-10 is engineered to comprise at least one nucleic acid sequence encoding a polypeptide.

19. The method of claim 16, wherein a transformed agrobacterium is suspended in a callus induction media.

20. The method of claim 16, wherein the injecting is performed at a wound site on the wounded plant.

21. A stable transformed plant obtained by the method of any of claims 11-15, wherein the stable transformed plant comprises all or part of the vector.

22. A progeny plant or seed obtained from the plant of claim 21, wherein the progeny plant or seed comprises all or part of the recombinant DNA construct.

23. A plant cell of a recalcitrant plant stably transformed with a recombinant DNA construct comprising at least one DR DNA sequence that encodes PLT5 or WIND1, and, optionally, a gene of interest.

24. The plant cell of claim 23, wherein the recombinant DNA construct comprises, as operably linked components, an optional first expression cassette which comprises a nucleic acid sequence encoding at least one selectable marker, an optional second expression cassette which comprises a cloning site, a third expression cassette which comprises the at least one DR DNA sequence that encodes PLT5 or WIND1, and an optional fourth expression cassette comprising a gene of interest.

25. The plant cell of claims 23 and 24, wherein the recalcitrant plant is snapdragon, B. rapa cabbage, or tomato.

26. A plant regenerated from the plant cell of any of claims 23-25.

27. The vectors of any of claims 1-10, wherein PLT is PLT5, and is optionally PLT5 cloned from Arabidopsis.
